(19) United States
(12) Patent Application Publication
(10) Pub. No.: US 2021/0369788 A1
(43) Pub. Date: Dec. 2, 2021

(54) PRODUCTION OF CANINE PANCREATIC ISLETS FROM AN IMMATURE PANCREAS

(71) Applicant: ANIMAL CELL THERAPY - ACT, PARIS (FR)

(72) Inventor: Paul CZERNICHOW, Paris (FR)

(73) Assignee: ANIMAL CELL THERAPY - ACT, PARIS (FR)

(21) Appl. No.: 16/762,829

(22) PCT Filed: Nov. 8, 2018
(86) PCT No.: PCT/EP2018/080668

§ 371 Date: May 8, 2020

(30) Foreign Application Priority Data
Nov. 8, 2017 (EP) 17306547.5

(51) Int. Cl. A61K 35/39; C12N 5/071; A61K 47/69

(52) U.S. Cl. CPC A61K 35/39; C12N 2509/00; A61K 47/6927; C12N 5/0677

(57) ABSTRACT

The present invention relates to an in vitro method for preparing and producing canine pancreatic islets from immature pancreatic tissue. Such islets express, produce and secrete insulin upon glucose stimulation. The invention further encompasses canine pancreatic islets obtainable according to the present method, islet population of said islets and compositions comprising said islets. It also relates to transduced canine pancreatic islets, or tumours or cells derived thereof. The present invention also concerns the use of said canine pancreatic islets or cells derived thereof for treating a canine pancreatic disorder, such as canine diabetes, or for diagnosing canine diabetes.

The present invention relates to a method for producing canine pancreatic islets (islets of Langerhans) in vitro from pancreatic tissue. It particularly concerns producing insulin and glucagon-secreting islets from pancreas obtained during the pre-natal, the neonatal or the non-adult period. The invention further encompasses canine pancreatic islets obtainable according to the present method, compositions comprising said islets, and applications thereof. The invention also concerns a method for transducing canine pancreatic islets with an immortalising gene and transduced islets obtained thereof. The invention further concerns a method for producing canine pancreatic beta cells from said canine pancreatic islets. The present invention also relates to the use of said canine pancreatic islets, of said transduced canine pancreatic islets, or of cells derived thereof, for treating a canine pancreatic disorder, such as canine diabetes, or for diagnosing canine diabetes. It also relates to methods of diagnosis of canine diabetes using said canine pancreatic islets, said transduced canine pancreatic islets, said canine pancreatic beta cells, or tumours or cells derived thereof.

### BACKGROUND OF THE INVENTION

Canine Diabetes, a Common Condition without an Ideal Treatment

The prevalence of canine diabetes, and diabetes in pet animals in general, has only been studied in recent years, especially at the epidemiological level. Nonetheless, the prevalence of animal diabetes increases, as in humans. Veterinarians estimate that the frequency of canine diabetes has tripled in 30 years in Europe and the USA. The causes of dog diabetes, however, have not been further characterized. As a consequence, canine diabetes is often diagnosed late in the disease course.

The most common form of diabetes in dogs resembles type 1 diabetes in humans, although other types of diabetes have also been described in dogs (Nelson and Reusch, 2014; Rand et al., 2004; Bonnet et al., 2010; Catchpole et al., 2005; Shield et al, 2015; Ahlgren et al., 2014; Davison et al., 2008; Kennedy et al., 2006; Gale, 2005; O'Kell et al., 2017).

Only one effective treatment, consisting in daily insulin injections, is available for all types of diabetes in dogs. Typically, a dog will receive a dose of about 1 Insulin Units (IU)/kg once per day (Davison et al., 2005). Such a treatment represents a significant financial burden and results in a substantial deterioration in the quality of life (Niessen et al., 2012).

In this context, there is a need for new, more effective and less heavy treatment. In this respect, cell therapy is clearly advantageous, as it may offer nearly unlimited source of either pluripotent or differentiated cells, that have the potential to be highly compatible with the animal to be treated.

### Cell Therapy and Veterinary Medicine

The treatment of chronic diseases or of injuries of domestic animals by cell therapy is already implemented in the veterinary field.

For example, treatments using stem cells isolated from fat tissue (adipose), collected on the domestic animal to be treated, have been recently developed (U.S. Pat. No. 6,777,231 B1, U.S. Pat. No. 6,429,013 B1). These adipose-derived stem cells are administered to diseased or damaged cartilages, tendons and joints of the domestic animal to be treated, and are intended to regenerate the damaged tissue (for example VetStem Regenerative Cells: VSRC™, developed by the company “Vet-Stem Biopharma”).

However, these stem cell-based therapies have not been applied to diabetes and other endocrine disorders.

In the field of diabetes, advances in cell therapy remain modest despite a developing interest in therapy “replacement” for the pet animals, mainly dogs and cats. In particular, pet animal organ harvesting networks have been developed, leading to the creation of organ libraries. Pancreatic islets (also called islets of Langerhans) can be collected from donated animal pancreases. Methods for isolating pancreatic islets from pancreas of adult dogs that are suitable for transplant have been described (Woolcott et al., 2012, U58735154 B2). The transplant of such pancreatic islets is intended to replace insulin injections in grafted compatible diabetic animals, by restoring physiological pancreatic islet functions (for example Kanslet™ developed for cats and dogs by LIKARDA LLC).

This important progress in animal diabetes therapy is however limited by several outstanding issues. In particular, pancreatic islets isolated by such methods can neither be maintained nor be expanded in vitro. Therefore, pancreatic islets isolated by such methods need to be stored by cryopreservation for preserving their endocrine functions before being transplanted to compatible acceptor diabetic animal (U.S. Pat. No. 8,735,154 B2). Moreover, these frozen pancreatic islets cannot be expanded. Thus, the amount of islets available is strictly dependent on the amount collected on each organ.

Attempts to maintain and expand ovine pancreatic islets in vitro have been described. However, in all cases, pancreatic islets lost their ability to produce insulin after a few days in culture. Islet-like cell clusters (ICC) prepared from foetal sheep pancreases lost their ability to secrete insulin after less than six days in culture (Tuch et al., 1996). Similarly, insulin secretion by foetal sheep pancreas small explants was totally lost in less than ten days of culture, and could not be restored (Tuch and Madrid, 1996).

To date, there is no method available for generating and maintaining functional canine pancreatic islets in vitro.

In a first step towards developing a cell therapy of canine pancreatic disorders, such as canine diabetes, it would thus be extremely beneficial to have a method allowing to easily and rapidly generate large quantities of canine pancreatic islets which can be maintained and expanded in vitro. Moreover, it would be extremely useful to have canine pancreatic islets which can be maintained and expanded in vitro and that are adapted for transplant or cell therapy.

### Pancreas Physiology and Pancreatic Beta Cells

The mammal mature pancreas contains two types of tissue: exocrine tissue composed of acinar cells that produce enzymes secreted via the pancreatic ducts into the intestine (e.g., carboxypeptidase-A) and endocrine tissue, also known as endocrine islets, including pancreatic islets (or islets of Langerhans), composed of cells that produce hormones such as insulin (beta cells), glucagon (alpha cells), somatostatin (delta cells) and pancreatic polypeptide (PP cells).

The ontogeny of the endocrine pancreas during foetal life and the structure of the islets of Langerhans in the adult have been quite extensively studied in mice, rats and humans (Steiner et al., 2010; Kim A et al., 2009; Pictet et al., 1972), but not in other mammals. Dog pancreas development was recently described in an immunocytochemical study (Bricout-Neveu et al., 2017). This study has shown that the key morphological events of the pancreatic development described in mice and humans also occurs in dogs. However, this study has shown that the morphologically mature endocrine structures were not observed until early postnatal life in dogs (Bricout-Neveu et al., 2017). This ontogenic pattern of the dog pancreas development is different from the ontogenic pattern of the human pancreas development. For example, the beta cells appear at the beginning of the second trimester of gestation in human. In contrast, this study has shown that beta cells appear later in dog development: they are only visible at mid gestation. Small islet-like structures can be observed only a few days before delivery in dog (Bricout-Neveu et al., 2017).

Importantly, the functional maturation of the canine hormone-secreting pancreatic tissues (or endocrine tissue) has not been described. In particular, the hormone-secreting capacity of the canine pancreas throughout development has not been described, thus preventing the development of successful methods of establishing and maintaining functional canine pancreatic islets.

Yet, generation of functional canine pancreatic islets in large amount represents an important objective, because such pancreatic islets could be used for transplant or cell therapy for canine diabetes, as explained above. In addition, such pancreatic islets would also be useful for screening new drugs that can modulate canine beta cell function and that are adapted for canine diabetes treatment.

Thus, there is a strong need for an efficient, reliable and reproducible method for generating functional canine pancreatic islets.

The present invention addresses this need.

### DESCRIPTION

### Method for Producing Canine Pancreatic Islets

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The practice of the present invention employs, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook et al, 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction, (Mullis et al, ed., 1994); A Practical Guide to Molecular Cloning (Perbal Bernard V., 1988); Phage Display: A Laboratory Manual (Barbas et al., 2001). Enzymatic reactions and purification techniques are performed according to manufacturer's specifications, as commonly accomplished in the art or as described herein.

The present inventors have developed an innovative approach for the efficient de novo generation of canine pancreatic islets. Such canine pancreatic islets exhibit all the functional and physiological properties of native in vivo canine pancreatic islets, and may be used in cell therapy.

In a first aspect, the invention provides a method for producing canine pancreatic islets. In particular, the invention is directed to a method for producing dog pancreatic islets.

As used herein, a “pancreatic islet” or “islet of Langerhans” or “pancreas islet” (these terms are synonymous in the context of the present application and should thus be construed to convey the same meaning) is a cluster of pancreatic cells that contains the endocrine (or hormone-producing) cells. In the context of the present application the pancreatic islet contains at least two pancreatic alpha cells and at least two pancreatic beta cells. Preferably, the pancreatic islet contains at least five pancreatic alpha cells and at least five pancreatic beta cells, more preferably at least ten pancreatic alpha cells and at least ten pancreatic beta cells, even more preferably, at least a hundred pancreatic alpha cells and at least hundred pancreatic beta cells.

As used herein, the term “endocrine cell” or “pancreatic endocrine cell” or “pancreas endocrine cell” refers to a cell derived or obtained from the pancreas and that produces at least one pancreatic hormone. Pancreatic hormones include, in particular, insulin (produced by beta cells), glucagon (produced by alpha cells), somatostatin (produced by delta cells) and pancreatic polypeptide (produced by PP cells). In the context of the present application, the endocrine cell is a beta cell, an alpha cell, a delta cell or a PP cell.

The term “canine” or “canine animal” as used herein refers to any animal member of the Canidae family. The Canidae family includes, but is not restricted to, any race of wolves (Canis lupus), dogs (species: Canis lupus familiaris), dingos (Canis lupus), coyotes (genus Canis), lycaons (genus Lycaon), foxes (genus Canis, Cerdocyon, Dusycyon, Lycalopex, Otocyon, Drocyon, Vulpes) and jackals (genus Canis).

A “canine pancreatic islet” or “canine islet of Langerhans” “canine pancreas islet” is a pancreatic islet of canine origin. Thus, a “canine pancreatic islet” is a pancreatic islet obtained or derived from the pancreas of any member of the Canidae family. Similarly, as used herein, a “dog pancreatic islet” or “dog islet of Langerhans” or “dog pancreas islet” is a pancreatic islet of dog origin. Thus, a “dog pancreatic islet” is a pancreatic islet obtained or derived from the pancreas of a dog.

As used herein, an “alpha cell”, “pancreatic alpha cell” or “pancreas alpha cell” (these terms are synonymous in the context of the present application and should thus be construed to convey the same meaning) is a cell of the islets of Langerhans of the pancreas which secretes the glucagon hormone.

A “canine pancreatic alpha cell” or “canine pancreas alpha cell” or “canine alpha cell” is an alpha cell of canine origin. Similarly, as used herein, a “dog pancreatic alpha cell” or a “dog pancreas alpha cell” or a “dog alpha cell” is an alpha cell of dog origin.

As used herein, a “beta cell”, “pancreatic beta cell” or “pancreas beta cell” (these terms are synonymous in the context of the present application and should thus be construed to convey the same meaning) is a cell of the islets of Langerhans of the pancreas which secretes the insulin hormone in response to glucose and other secretagogues.

A “canine pancreatic beta cell” or “canine pancreas beta cell” or “canine beta cell” is a beta cell of canine origin. Similarly, as used herein, “dog pancreatic beta cell” or “dog pancreas beta cell” or “dog beta cell” is a beta cell of dog origin.

The term “pancreatic tissue” or “pancreas tissue” as used herein refers to a tissue obtained or derived from the pancreas; likewise, the term “pancreatic cells” refers herein to cells obtained or derived from pancreas. The term “canine pancreatic tissue” as used herein refers to a pancreatic tissue of canine origin. Thus, a “canine pancreatic tissue” is a pancreatic tissue obtained or derived from the pancreas of any member of the Canidae family; likewise, the term “canine pancreatic cells” or “canine pancreas cells” refers herein to cells obtained or derived from pancreas of any member of the Canidae family.

As used herein, the term “endocrine pancreas tissue”, “endocrine pancreas” or “endocrine pancreas structure”, also known as endocrine islets, refers to a pancreas tissue which comprises endocrine cells that produce hormones such as insulin (beta cells), glucagon (alpha cells), somatostatin (delta cells) or pancreatic polypeptide (PP cells). In the context of the present application, the endocrine pancreas tissue or the endocrine pancreas structure comprises at least one pancreatic islets comprising at least one endocrine cells. In particular, the endocrine pancreas tissue or the endocrine pancreas structure in the context of the present application comprises at least one pancreatic islet, wherein said at least one islet comprises at least two pancreatic alpha cells and at least two pancreatic beta cells as defined above. In the context of the application, the endocrine pancreas tissue comprises at least one endocrine pancreas structure.

The present inventors have devised a new and innovative strategy for de novo generating canine islets from immature canine pancreatic tissue materials.

They have surprisingly and unexpectedly discovered that neogenesis of canine islets can be achieved in vitro using pancreatic endocrine cells collected from immature canine pancreatic tissue at a specific developmental stage. The inventors were able to obtain de novo functional canine pancreatic islets, also called neo-islets, which are capable of stably producing canine insulin and glucagon. These neo-islets are capable of responding to glucose stimulation. Notably, glucose stimulation induces a significant and reproducible increase of insulin secretion by these neo-islets. In addition, a decrease in blood glucose concentration is observed in animals grafted with these neo-islets. Thus, the canine pancreatic neo-islets obtained by the inventors have all the functional and physiological properties of native in vivo canine pancreatic islets. Importantly, the canine pancreatic neo-islets obtained by the inventors can be efficiently and rapidly amplified in vitro while retaining all the functional and physiological properties of native in vivo canine pancreatic islets. These functional canine pancreatic neo-islets can thus be efficiently and rapidly amplified in vitro to large amounts for therapeutic, testing or diagnostic use in a reproducible way. This is particularly unexpected and inventive because the methods for isolating pancreatic islets from pancreases obtained from canine or other mammals that are described in the art do not allow maintenance and/or amplification of functional pancreatic islets isolated thereof (Tuch et al., 1996; Woolcott et al., 2012; U.S. Pat. No. 8,735,154 B2). The only possibility, that was known in the art, to maintain such pancreatic islet was cryopreservation.

Accordingly, the present invention relates to a method for specifically establishing and amplifying canine pancreatic islets from canine pancreatic tissues.

Several batches of canine pancreatic neo-islets have been thus independently generated. All of them stably express canine insulin and glucagon, and are capable of producing and secreting both canine insulin and glucagon. These canine pancreatic neo-islets are capable of reproducibly responding to glucose stimulation and regulating blood glucose levels and are therefore fully functional.

This opens considerable perspectives in the veterinary use of pancreatic islets in the treatment of canine pancreatic disorders, such as diabetes.

In particular, the present inventors have surprisingly and unexpectedly discovered that the functional pancreatic islets obtained by this new process are capable of developing canine endocrine pancreas-like tissue after transplant under the kidney capsule of an animal, in a reproducible manner. After grafting, a significant amount of canine insulin is detected in the blood of the grafted animals. In addition, a decrease in blood glucose concentration is observed in grafted animals. The endocrine pancreas-like tissues thus developed have all the functional and physiological properties of native in vivo canine endocrine pancreas. This new process for obtaining insulin and glucagon-secreting islets by the method of the invention thus offers an abundant source of stable and functional canine pancreatic islets that can be used in cell or grafting therapy for the treatment of canine pancreatic disorders, such as diabetes.

The canine pancreatic islets obtained by the method of the invention can also be efficiently used to detect the presence of auto-antibodies found in sera of diabetic canines and thereby have a great potential for diagnosis of canine diabetes.

The present invention thus provides for the first time a method for in vitro generation and amplification of functional canine pancreatic islets.

The present inventors have surprisingly and unexpectedly discovered that de novo generation and amplification of functional canine pancreatic islets can be achieved in vitro using canine pancreatic endocrine cells obtained from an immature canine pancreas. The inventors have surprisingly found that, when canine pancreatic endocrine cells obtained from an immature canine pancreas are incubated in a culture medium, functional canine pancreatic islets, called neo-islets, can be reproducibly and efficiently generated and amplified. The canine pancreatic neo-islets thus obtained display the spherical morphology of in vivo native pancreatic islets. The inventors have shown that the neo-islets are capable of stably expressing and producing both insulin and glucagon and are capable of regulating blood glucose levels and of responding to glucose stimulation. Indeed, the inventors have surprisingly found that, when the glucose concentration in the culture medium is comprised between 4 mM to 30 mM, the canine pancreatic neo-islets are capable of secreting insulin in the culture medium. After grafting the canine pancreatic neo-islets under the kidney capsule of a host animal, a significant amount of canine insulin is detected in the blood of the grafted animals. In addition, a decrease in blood glucose concentration is observed in grafted animals. The canine pancreatic islets thus obtained have all the functional and physiological properties of native in vivo canine pancreatic islets. In particular, they are capable of producing canine insulin and glucagon. In addition, they are capable of responding to glucose stimulation by secreting insulin.

In a first embodiment, the invention is directed to a method for preparing canine pancreatic islets, said method comprising the steps of:
a) obtaining canine pancreatic endocrine cells from an immature canine pancreas or a portion thereof; and
b) incubating the pancreatic endocrine cells of step a) in an appropriate culture medium comprising glucose at a concentration comprised between 4 mM to 30 mM.

The present inventors have shown that incubating the pancreatic endocrine cells obtained of step a) in an appropriate culture medium comprising glucose at a concentration comprised between 4 mM to 30 mM allows pancreatic islets to develop and amplify.

Thus, in one embodiment, the invention is directed to a method for preparing canine pancreatic islets, said method comprising the steps of:

a) obtaining canine pancreatic endocrine cells from an immature canine pancreas or a portion thereof; and

b) incubating the pancreatic endocrine cells of step a) in an appropriate culture medium comprising glucose at a concentration comprised between 4 mM to 30 mM, allowing pancreatic islets to develop and/or generate and/or amplify.

In one embodiment, the invention is directed to a method for preparing canine pancreatic islets, said method comprising the steps of:

a) obtaining canine pancreatic endocrine cells from an immature canine pancreas or a portion thereof;

b) incubating the pancreatic endocrine cells of step a) in an appropriate culture medium comprising glucose at a concentration comprised between 4 mM to 30 mM for a sufficient period of time to develop and/or generate and/or amplify pancreatic islets.

As used herein, the term “immature pancreas” refers to a foetal pancreas, a neonatal pancreas or a non-adult pancreas that have gone through a first endodermal differentiation and that does not have all the morphological, functional and physiological properties of a fully mature adult pancreas. In particular, a pancreas that does not have all the morphological, functional and physiological properties of a fully mature adult pancreas may be a pancreas which presents a ratio of beta cells to alpha cells that is different to the ratio of beta cells to alpha cells observed in an adult pancreas of the same species. In particular, the ratio of beta cells to alpha cells of an immature pancreas is lower than the ratio of beta cells to alpha cells of a mature pancreas (such as an adult pancreas). A pancreas that does not have all the morphological, functional and physiological properties of a fully mature adult pancreas may be pancreas which present a number and/or distribution of alpha cells, beta cells and/or pancreatic islets that is different from the distribution of alpha cells and/or beta cells observed in an adult of the same species. In particular, an immature pancreas has pancreatic islets that are less in number and/or more scattered compared to a mature pancreas.

As used herein, the term “first endodermal differentiation” refers to a stage of foetal development wherein undifferentiated embryonic cells differentiate towards the endoderm lineage.

The term “immature canine pancreatic islets” or “immature canine pancreas islets” as used herein refers to pancreatic islets which may be obtained or derived from an immature pancreas of any member of the Canidae family.

The immature canine pancreas may be obtained from any canine animal as defined above. Preferably, the canine animal is a dog (species: Canis lupus familiaris).

Preferably, the immature canine pancreas is an immature dog pancreas.

In a preferred embodiment, the pancreatic endocrine cells of step a) comprise at least one beta cell. More preferably, the pancreatic endocrine cells of step a) comprise at least one beta cell and at least one alpha cell. Preferably, the pancreatic endocrine cells of step a) comprise beta cells and alpha cells. The pancreatic endocrine cells of step a) may further comprise at least one delta cell and at least one a PP cell. In one embodiment, the pancreatic endocrine cells of step a) further comprise precursor endocrine cells.

The canine pancreatic neo-islets thus obtained by the method of the invention have all the functional and physiological properties of native in vivo canine pancreatic islets. In particular, they are capable of producing canine insulin and glucagon. They are capable of responding to glucose stimulation and of regulating glucose blood levels in a grafted animal. This is particularly unexpected and inventive because the methods for isolating pancreatic islets from canine pancreas that are described in the art do not allow maintenance and/or amplification of pancreatic islets isolated thereof (Woolcott et al., 2012, U.S. Pat. No. 8,735,154 B2). The only possibility, that was known in the art, to maintain such pancreatic islet was cryopreservation. With the method of the invention, not only canine pancreatic islets can be quantitatively produced and maintained, but their physiological and functional properties are preserved. The terms “neo-islet”, “pancreatic neo-islets”, “functional neo-islets” or “canine pancreatic neo-islets” as used herein refers to the canine pancreatic islets obtained by the method of the invention.

In the context of the present application, pancreatic islets generate if pancreatic islets as defined above are formed de novo in the culture medium during step b) of incubating the pancreatic endocrine cells of step a). Pancreatic islets amplify (or expand, both terms have the same meaning in the context of the present application) if pancreatic islets as defined above multiply in the culture medium during step b) of incubating the pancreatic endocrine cells of step a). Pancreatic islets develop if pancreatic islets as defined above generate and multiply in the culture medium during step b) of incubating the pancreatic endocrine cells of step a).

In one embodiment, the endocrine cells of step b) are incubated for a sufficient period of time allowing the pancreatic islets to develop. A sufficient period of time to develop and/or generate and/or amplify pancreatic islets is at least two days, preferably at least 3 days, more preferably at least 4 days. Preferably, the endocrine cells of step b) are incubated for at least 2 days, more preferably at least 3 days, even more preferably at least 4 days, even more preferably at least 5 days, even more preferably at least 6 days, even more preferably at least 7 days, even more preferably at least 2 weeks, even more preferably at least 3 weeks, even more preferably at least 4 weeks, to allow the pancreatic islets to develop as defined above.

In one embodiment, the method of the invention comprises the further step c) of in the culture medium for a sufficient period of time.

As of today, the development of the canine pancreas has not been extensively studied, preventing, at least in part, the successful generation of canine pancreatic islets. The present inventors were the first one to describe the early morphological development of the canine endocrine pancreas (Bricout-Neveu et al., 2017).

Notably, the present inventors were the first to show that canine insulin positive cells begin to emerge at mid gestation, around 30 days of the foetal life. The present inventors have notably shown that small islet-like structures can be observed a few days before delivery in dog. The morphologically-mature endocrine pancreas structures, positive for both insulin and glucagon expression, however, are observed only in the early post-natal life in the dog (Bricout-Neveu et al., 2017). Yet, despite the fact that very few islets structures are present in the canine foetal pancreas, the present inventors were able to successfully and reproducibly generate functional pancreatic islets in vitro, from immature canine pancreatic tissue obtained in the last third of gestation. This is particularly surprising and unexpected because the canine immature pancreas is very small compared to the pancreas of the adult canine or the pancreas of the foetus or neonates of bigger mammals, such as the ovine and porcine pancreas. Isolation of pancreatic islets from surrounding acinar cells and undifferentiated cells in the canine foetus is particularly difficult. The present inventors were able to overcome this difficulty. In particular, the inventors have shown that the canine pancreatic islets do not need to be surgically isolated from the surrounding foetal pancreatic cells and that the incubation of minced foetal canine pancreatic tissue comprising endocrine cells in a culture medium allows to selectively isolate the foetal pancreatic islets and to generate neo-islets. The canine pancreatic neo-islets thus obtained have all the functional and physiological properties of native in vivo canine pancreatic islets. In particular, they are capable of producing canine insulin and glucagon, of regulating glucose blood levels in a grafted animal and of responding to glucose stimulation.

Thus, in one embodiment of the method of the invention, the pancreatic endocrine cells of step a) of the method of the invention are obtained from at least one foetal canine pancreas which has been obtained from a subject in the last third of gestation.

Preferably, the pancreatic endocrine cells of step a) of the method of the invention are recovered from a foetal canine pancreas removed at days 40 to 60 post conception (pc). Yet preferably, the pancreatic endocrine cells are collected from a foetal canine pancreas removed at days 45 to 60 post conception. Advantageously, the pancreatic endocrine cells are recovered from a foetal canine pancreas removed at days 45 to 60 post conception. In a preferred embodiment, the pancreatic endocrine cells according to the invention are obtained from a foetal canine pancreas removed at days 50 to 60 post conception, more preferably from a foetal canine pancreas removed at days 50, 51, 52, 53, 54, 55 56, 57, 58, 59 or 60 post conception. Yet preferably, the pancreatic endocrine cells of step a) of the method according to the invention are recovered from a foetal canine pancreas removed at days 52 to 58 post conception, more preferably from a foetal canine pancreas removed at days 53 to 57 post conception. Indeed, the inventors have shown that higher amounts of functional pancreatic neo-islets are obtained using the method of the invention when the foetal canine pancreas is obtained at days 40 to 60 post conception and that this amount is particularly high when the foetal canine pancreas is obtained at days 50 to 55 post conception, compared to when the foetal canine pancreas is obtained prior to day 40 post conception, after an incubation in the culture medium of step b) in comparable conditions (for instance when incubating the same amount of pancreatic endocrine cells obtained in step a) or of the same age, for the same period of time (such as 6 to 14 days), and/or in the same culture medium and/or at a same temperature). Accordingly, as used herein, the term yield refers to the amount of pancreatic neo-islets obtained by the method of the invention, after the incubation of a determined amount of pancreatic endocrine cells obtained in step a) from a pancreas obtained at a determined stage of development, in the culture medium of step b), in determined conditions (including time length of culture, composition of culture medium, temperature).

In one embodiment, from 10000 to 100000 pancreatic endocrine cells obtained in step a) are incubated in step b). Preferably, from 20000 to 90000 pancreatic endocrine cells obtained in step a) are incubated in step b), more preferably from 30000 to 80000 pancreatic endocrine cells, even more preferably 40000 to 70000 pancreatic endocrine cells obtained in step a) are incubated in step b). From 40000 to 80000 pancreatic endocrine cells may be obtained from six to eight 55-day-old foetal pancreases (at day 55 post conception).

The amount of endocrine cells and of pancreatic neo-islets obtained by the method of the invention may be determined using at least one method known in the art. For instance, the amount of endocrine cells may be measured by counting the total number of cells obtained at the end of step a), for example by counting the number of cells visible under the microscope. The total number of endocrine cells may be evaluated by determining the density of cells obtained at the end of step a), for example by determining the optical density. For instance, the amount of islets may be measured by counting the total number of islets-like structures obtained at the end of step b), for example by counting the number of islets visible under the microscope. The total number of islets may be evaluated by determining the density of islets obtained at the end of step b), for example by determining the optical density. The amount of endocrine cells or of pancreatic neo-islets obtained by the method of the invention may also be determined by measuring the expression levels of insulin and/or glucagon of said cells or islets, or the in vitro secretion levels of insulin and/or glucagon by the endocrine cells or the neo islets using methods known in the art, for example using immunocytochemical methods.

Methods for measuring and/or determining the expression levels of insulin and/or glucagon or the in vitro secretion levels of insulin and/or glucagon are generally known to those skilled in the art and has routinely relied on methods developed to measure human insulin or glucagon. Methods for measuring and/or determining the level of expression of insulin include, for example PCR-based techniques (PCR for polymerase chain reaction), including quantitative PCR or RT-PCR, hybridization with a labeled nucleic acid probe, such as by northern blot (for mRNA) or by Southern blot (for cDNA), serial analysis of gene expression (SAGE) method and its derivatives, such as longSAGE, superSAGE, deepSAGE; tissue chips (also known as TMAs: tissue microarrays). The tests usually used with tissue chips comprise immunohistochemistry and fluorescent in-situ hybridization. For mRNA analysis, tissue chips can be paired with fluorescent in-situ hybridization. It is also possible to use RNA or complementary DNA sequencing, such massive parallel sequencing to determine the quantity of mRNA or cDNA in the sample (RNA-Seq or whole transcriptome shotgun sequencing), transcriptome analysis, nucleic acid microarrays. Methods for measuring and/or determining the level of expression of insulin at the protein level, and/or the in vitro secretion levels of insulin and/or glucagon include, for example, mass spectrometry, biochemical tests, including immunological tests such as, for example, traditional immunological detection tests (enzyme-linked immunosorbent assay or ELISAs and ELISPOT assays), or such as, for example, immunological tests employing techniques involving transfer of proteins on a support, such as the slot blot (also called dot blot) or the western blot. It is possible, for example, to employ enzymatic assays, protein microarrays, antibody microarrays or tissue microarrays coupled with immunohistochemistry. Among other techniques that can be used are BRET or FRET techniques, methods of microscopy or histochemistry, including in particular methods of confocal microscopy and electron microscopy, methods based on the use of one or more excitation wavelengths and a suitable optical method, such as an electrochemical method (voltammetry and amperometry), atomic force microscopy, and methods of radio frequency, such as multipolar resonance spectroscopy, confocal and non-confocal, detection of fluorescence, luminescence, chemiluminescence, absorbance, reflectance, transmittance, and birefringence or refraction index (for example, by surface plasmon resonance, by ellipsometry, by a resonant mirror method, etc.), flow cytometry, by radioisotope or magnetic resonance imaging, analysis by polyacrylamide gel electrophoresis (SDS-PAGE); by HPLC-mass spectrophotometry, by liquid chromatography/mass spectrophotometry/mass spectrometry (LC-MS/MS). All these techniques are well-known to the skilled person and it is not necessary to detail them herein.

The present inventors were also able to successfully and reproducibly generate functional pancreatic neo-islets in vitro, from immature canine pancreatic tissue obtained in the neonatal period. Thus, in one embodiment of the method of the invention, the pancreatic endocrine cells of step a) are recovered from at least one canine pancreas which has been obtained from a subject in the neonatal period.

The terms “neonatal period” or “early postnatal period” as used herein refers to the interval from birth until weaning. Accordingly, a neonatal pancreas is a pancreas which has been obtained from a subject during the interval from the day of birth to the first day of weaning.

In one embodiment of the method of the invention, the pancreatic endocrine cells according to the invention are recovered from at least one canine pancreas which has been obtained from a non-adult subject. As used herein, a “non-adult canine subject” or “non-adult canine” is a canine less than 3-month-old, preferably less than 2-month-old, more preferably less than 1.5-month-old, even preferably less than 1-month-old.

The pancreatic endocrine cells of step a) can be recovered from at least one foetal canine pancreas. The pancreas endocrine cells of step a) can also be obtained from at least one neonatal or non-adult canine pancreas. The pancreatic endocrine cells according to the invention can be recovered from the whole foetal, neonatal or non-adult canine pancreas or only a portion of said pancreas. The pancreatic endocrine cells according to the invention can be recovered from one or more foetal, neonatal or non-adult canine pancreases or mixtures thereof.

In one embodiment, the pancreatic tissue has been frozen after being harvested. In another embodiment, the pancreatic tissue used in the method of the invention is fresh. Thus, according to that specific embodiment, the method of the invention comprises a step of harvesting the pancreatic tissue prior to step a).

In one embodiment, step a) of the method of the invention comprises a further step of mincing the immature pancreas or portion thereof. Step a) of the method of the invention can also comprise a further step of dissociating an immature canine pancreas or portion thereof by digesting with an appropriate enzyme in order to obtain canine pancreatic endocrine cells or canine pancreatic endocrine cell aggregates. In one embodiment, step a) of the method of the invention comprises a further step of mincing the immature pancreas or portion thereof followed by a further step of digesting the minced immature canine pancreas or portion thereof with an appropriate enzyme in order to obtain canine pancreatic endocrine cells or canine pancreatic endocrine cell aggregates. Said appropriate enzyme is preferably collagenase.

Accordingly, in one embodiment, the invention is directed to a method for preparing canine pancreatic islets, said method comprising the steps of:
a) obtaining canine pancreatic endocrine cells from an immature canine pancreas or a portion thereof, wherein said canine pancreatic endocrine cells are obtained by digesting the said immature pancreas or portion thereof with collagenase; and
b) incubating the pancreatic endocrine cells of step a) in an appropriate culture medium comprising glucose at a concentration comprised between 4 mM to 30 mM, allowing pancreatic islets to develop.

By “collagenase”, it is herein referred to an enzyme belonging to the matrix metalloprotease (MMP) family which is capable of breaking the peptide bonds in collagen. A collagenase according to the invention can be either of bacterial or animal origin. Bacterial collagenases differ from vertebrate collagenases in that they exhibit broader substrate specificity. Unlike animal collagenases, bacterial collagenase can attack almost all collagen types, and is able to make multiple cleavages within triple helical regions. Preferably, the collagenase of the invention is a bacterial enzyme; more preferably, it is an enzyme secreted by the anaerobic bacteria Clostridium histolyticum. In a preferred embodiment, the collagenase used in the invention is selected from the group consisting of collagenases Type I-S, Type IA, Type IA-S, Type II, Type II-S, Type IV, Type IV-S, Type V, Type V-S, Type VIII, Type XI and Type XI-S. In the most preferred embodiment, the collagenase of the invention is collagenase XI.

The concentration of the collagenase used to obtain canine pancreatic endocrine cells in the method of the invention is preferably inferior or equal to 7 mg/mL; more preferably, to 6 mg/mL; even more preferably, to 6 mg/mL; still more preferably, to 5 mg/mL; yet even more preferably, to 4 mg/mL. In the most preferred embodiment, said collagenase is used at 1 mg/mL. According to the invention, immature canine pancreatic tissue is dissociated with collagenase for at least 10 minutes; preferably for at least 15 minutes; more preferably at least 20 minutes; even more preferably at least 25 minutes; still more preferably at least 30 minutes; most preferably for 30 minutes at about 37° C. For dissociation to occur, the above-mentioned pancreatic tissues are preferably suspended in an appropriate medium comprising PBS+20% FCS. The collagenase dissociation reaction can be stopped by any appropriate mean, such as by diluting the collagenase reaction and/or by successive washes of the dissociated pancreas.

To this date, a method for in vitro generating, expanding and maintaining canine pancreatic islets that have all the functional and physiological properties of native in vivo canine pancreatic islets has not been described. The present inventors were the first one to show that neogenesis, expansion and maintenance of fully functional canine pancreatic neo-islets can be rapidly, easily and reproducibly achieved by incubating the pancreatic endocrine cells collected from an immature pancreas, as described above, in an appropriate culture medium. The inventors notably show that the yield of obtaining functional canine pancreatic neo-islets is particularly high when the collected canine pancreatic endocrine cells are incubated in a culture medium comprising glucose at a concentration comprised between 4 mM to 30 mM. Accordingly, step b) of the method of the invention comprises incubating the canine pancreatic endocrine cells obtained in step a) in an appropriate culture medium comprising glucose at a concentration comprised between 4 mM to 30 mM. Preferably, the glucose concentration in said appropriate culture medium is comprised between 4.5 mM to 25 mM, yet preferably between 5 mM to 20 mM, even more preferably between 5 mM to 15 mM, even more preferably between 5.5 mM to 13 mM, even more preferably between 5.5 mM to 12 mM, even more preferably between 5.5 mM to 11 mM. In a preferred embodiment, the glucose concentration in said appropriate culture medium is comprised between 5.5 mM to 11 mM. Preferably the glucose concentration in said appropriate culture medium is 4.5 mM, 5 mM, 5.5 mM, 6 mM, 6.5 mM, 7 mM, 7.5 mM, 8 mM, 9 mM, 10 mM, or 11 mM.

Thus, a method for generating, expanding and maintaining fully functional canine pancreatic islets in vitro could not be implemented so far at least also because the proper conditions for incubating said canine pancreatic endocrine cells were not known.

Accordingly, in one embodiment, the invention is directed to a method for preparing canine pancreatic islets, said method comprising the steps of:
a) obtaining canine pancreatic endocrine cells from an immature canine pancreas or a portion thereof, optionally wherein said canine pancreatic endocrine cells are obtained by digesting the said immature pancreas or portion thereof with an appropriate enzyme, preferably collagenase; and
b) incubating the pancreatic endocrine cells of step a) in an appropriate culture medium comprising glucose at a concentration comprised between 4 mM to 30 mM, preferably at a concentration comprised between 4.5 mM to 25 mM, yet preferably between 5 mM to 20 mM, even more preferably between 5 mM to 15 mM, even more preferably between 5.5 mM to 13 mM, even more preferably between 5.5 mM to 12 mM, even more preferably between 5.5 mM to 11 mM, allowing pancreatic islets to develop;

wherein the immature canine pancreas is preferably an immature dog pancreas, and preferably wherein the immature pancreas is a foetal pancreas (preferably the foetal pancreas is obtained at days 40 to 60 pc, more preferably at days 45 to 60 pc, even more preferably at days 50 to 60 pc, even more preferably at days 52 to 58 pc, yet preferably at days 53 to 57 pc), a neonatal pancreas, or a non-adult pancreas.

In one embodiment, the appropriate culture medium is a commercially available culture medium appropriate for growing mammalian cells, such as glucose-free RPMI (Roswell Park Memorial Institute medium), supplemented in glucose (to a concentration comprised between 4 mM to 30 mM). Said commercially available culture medium may be further supplemented in fetal calf serum, HEPES (N-2-hydroxyethyl piperazine-N′-2-ethane sulfonic acid buffer) and suitable antibiotics.

In one embodiment, the method of the invention further comprises the step c) of encapsulating the pancreatic islets of step b) in a device, preferably a protective device. Such protective device may protect the pancreatic islets from degradation or limit degradation of the pancreatic islets. Protection may be desired, for instance in case of prolonged storage and/or of transportation of the pancreatic islets. Protection of the pancreatic islets may also be desired if the administration or the transplant of the islets in a subject in need thereof is contemplated. In such case, the protective device may be designed to protect the islets against, or limit immune, bacterial and/or viral attacks after administration of transplant in said subject. The device may also be designed for allowing the diffusion of oxygen nutrients and secretagogues, including glucose, to the encapsulated cells and also permit the diffusion of insulin and/or glucagon out to the surrounding environment. The practical development of the device will be determined by a person skilled in the art in application of his general knowledge in the matter to obtain the desired level of protection.

Said protective device may for example comprise or consist of a semi-permeable membrane of high polymer, such as alginate (an unbranched binary copolymer of 1-4 linked β-D-mannuronic acid (M) and α-L-guluronic acid (G), of varying composition and sequential structure (MMM-blocks, GGGblocks and MGM-blocks)), poly ethylene glycol (PEG), poly-L-lysine (PLL), polysulphone (PSU), Polyvinyl alcohol (PVA), or agarose, or any mixture thereof. Such mixture includes for instance mixture of alginate and poly (ethylene glycol) (PEG) or mixture of alginate and poly-L-lysine (PLL), mixed or arranged in two or more successive layers. The protective device may also comprise a mesh reinforcement. Said protective device may also comprise or consist of one or more microcapsules, one or more microcapsules or a mixture thereof. In one embodiment, the protective device is a capsule.

Accordingly, in one embodiment, the invention is directed to a method for preparing canine pancreatic islets, said method comprising the steps of:
a) obtaining canine pancreatic endocrine cells from an immature canine pancreas or a portion thereof, optionally wherein said canine pancreatic endocrine cells are obtained by digesting the said immature pancreas or portion thereof with an appropriate enzyme, preferably collagenase;
b) incubating the pancreatic endocrine cells of step a) in an appropriate culture medium comprising glucose at a concentration comprised between 4 mM to 30 mM, preferably at a concentration comprised between 4.5 mM to 25 mM, yet preferably between 5 mM to 20 mM, even more preferably between 5 mM to 15 mM, even more preferably between 5.5 mM to 13 mM, even more preferably between 5.5 mM to 12 mM, even more preferably between 5.5 mM to 11 mM allowing pancreatic islets to develop; and
c) encapsulating the pancreatic islets of step b) in a device, preferably a protective device;

preferably wherein the immature canine pancreas is an immature dog pancreas, and preferably wherein the immature pancreas is a foetal pancreas (preferably the foetal pancreas is obtained at days 40 to 60 pc, more preferably at days 45 to 60 pc, even more preferably at days 50 to 60 pc, even more preferably at days 52 to 58 pc, yet preferably at days 53 to 57 pc), a neonatal pancreas, or a non-adult pancreas.

The pancreatic islets obtained by the method of the invention may be further expanded and maintained in vivo by sub-grafting said islets in a severe combined immunodeficiency (scid) non-human animal. Indeed, the present inventors have surprisingly discovered that grafting the canine pancreatic neo-islets obtained by the method described above, in a scid mouse results in the development in a fully mature and functional endocrine pancreas-like tissue, resulting in a significant production of canine insulin in the blood of the grafted animal and in a decrease in blood glucose concentration.

Accordingly, in one embodiment, the method of the invention comprises a further step c′) performed after step b), and optionally after step c) of encapsulation, comprising the steps of:
c′1) introducing the canine pancreatic islets obtained in b) or the encapsulated canine pancreatic islets of step c) into the kidney capsule of a first severe combined immunodeficiency (scid) non-human animal;
c′2) allowing the canine pancreatic islets to develop endocrine pancreas-like structures;
c′3) micro-dissecting the endocrine pancreas-like structures obtained in step c′2), and collecting the pancreatic islets thereof;
c′4) sub-transplanting the islets obtained in step c′3) into the kidney capsule of a second scid non-human animal;
c'5) allowing the sub-transplanted islets in step c′4) to develop and regenerate newly developed endocrine pancreas-like structures, wherein said newly developed insulinoma-like structures are enriched in insulin-producing pancreatic islets;
c′6) micro-dissecting the endocrine pancreas-like structures obtained in step c'5), and collecting the islets thereof;
c′7) optionally, sub-transplanting the islets obtained in step c′6) into the kidney capsule of a third non-human scid animal, hence allowing further enrichment and amplification of insulin-producing pancreatic islets; and
c′8) optionally repeating step c′4), c'5) and c′6) until the appropriate amount of insulin-producing islets is obtained.

A scid animal is an animal lacking T and B lymphocytes and failing to generate either humoral or cell mediated immunity. The scid non-human animal as referred herein can be selected among bovines, porcines, horses, sheep, goats, primates except humans, rodents such as mice, rats, hamsters. Said scid animal is preferably a scid mouse.

Said scid non-human animal can be a diabetic animal wherein diabetes is induced by chemical destruction of the beta cells. Beta cells can be chemically destructed by administering streptozotocine to said scid animal, according to methods known in the art. Said scid non-human animal can carry at least one other type of mutation leading to immunodeficiency. Said scid non-human animal can be a non-obese diabetic/severe combined immunodeficiency (NOD/scid) animal. A NOD/scid animal is an animal lacking T and B lymphocytes, which thus fails to generate either humoral or cell-mediated immunity. In a preferred embodiment, the streptozotocine-treated scid animal or the NOD/scid animal used in the method of the invention is a mouse. NOD/scid mice are known in the literature and are commercially available from suppliers such as Charles River or Jackson Laboratory. Preferably the streptozotocine-treated scid animal or the NOD/scid mouse used in the method of the invention is of any age of development, preferably sufficiently old so that a graft into the kidney capsule can be performed. Preferably, the streptozotocine-treated scid animal or the NOD/scid mice are about of the 2 to 15 weeks of development, more preferably to 6 to 8 weeks of development.

The above-defined method may include collecting the canine functional pancreatic islets obtained at step b) or step c′8), to form a homogenous islet population. The islet population can further be grown in vitro to establish a homogenous and functional canine pancreatic islet population.

The above method to prepare canine functional islets is particularly useful for preventing or treating a canine pancreatic disorder. The above method to prepare canine functional islets is also particularly useful for testing and screening candidate medicaments for treating canine pancreatic disorders, in vivo after graft in non-human animals, such as mice or rats, or in vitro.

In this regard, and in one embodiment, the above method can be put to practice to prepare large amount of canine functional pancreatic islets for therapeutic purposes, for testing and screening purposes as well as for in vitro diagnosis of canine diabetes allowing classification of diabetic animals in type 1 diabetes or other types of diabetes. With the above method, large amount of functional canine pancreatic islets can be efficiently obtained in vitro. Additionally, steps c′4), c′5) and c′6) can be repeated as many times as necessary to obtain large amount of endocrine pancreas-like structures and functional canine pancreatic islets and these islets may further be amplified in culture in vitro. In still another embodiment, the method for preparing canine pancreatic islets as described above is directed to the establishment of master banks for therapeutic or diagnostic purposes, such as cell therapy of canine pancreatic disorders, for testing and screening purposes. Thus, in one aspect, the present invention encompasses a method for the establishment of master cell banks for cell therapy of diabetes, comprising the step of obtaining canine pancreatic islets by the method of the invention.

Canine pancreatic islets, canine pancreatic islet populations and banks

In a second aspect, the invention is aimed at canine pancreatic islets obtainable by the above-described method.

In one embodiment, these canine pancreatic islets possess at least one of the following features:
presence of canine alpha cells;
presence of canine beta cells;
expression of canine-specific insulin; and
expression of canine-specific glucagon.

In one embodiment, the canine pancreatic islets of the invention possess all of the following features:
presence of canine alpha cells;
presence of canine beta cells;
expression of canine-specific insulin; and
expression of canine-specific glucagon.

Advantageously, said pancreatic islets further display at least one of the following features:
Carboxypeptidase-A negative;
transcriptional factor Pdx1 positive;
transcription factor MafA positive;
proconvertase Pcsk1 positive;
expression of Glucose transporter Glut2;
expression of Kcnj11 and Abcc8 coding for subunits of the potassium channel; and
expression of zinc transporter Znt8 (Slc30a8).

Preferably, the canine pancreatic islets of the invention are also positive to reaction with canine-specific anti-insulin, canine-specific anti-glucagon, anti-GAD and/or anti-IA2 antibodies.

In one embodiment, the canine pancreatic islets of the invention are capable of secreting canine specific insulin in response to glucose stimulation. Accordingly, the canine pancreatic islets are capable of secreting increasing amounts of insulin in correlation with increasing concentration of glucose, in vitro and/or in vivo. Advantageously, the canine pancreatic islets of the invention are capable of secreting canine specific glucagon in response to glucose stimulation. According to this embodiment, the canine pancreatic islets are capable of secreting increasing amounts of glucagon in correlation with decreasing concentration of glucose, in vitro and/or in vivo. Advantageously, the canine pancreatic islets of the invention have all the functional and physiological properties of native in vivo canine pancreatic islets. In particular, they are capable of producing canine insulin and glucagon.

The canine pancreatic islets of the invention can be maintained and grown in culture in an appropriate culture medium comprising glucose at a concentration comprised between 4 mM to 30 mM as defined above. Indeed, the inventors were the first to show that the canine pancreatic islets grown and maintained in such medium, are capable of stably, efficiently and homogenously producing canine insulin. Thus, the invention also contemplates a culture comprising the above-described canine pancreatic islets in culture an appropriate culture medium comprising glucose at a concentration comprised between 4 mM to 30 mM as defined above. This culture allows to expand and to establish homogeneous canine pancreatic islet populations.

The canine pancreatic islets of said populations of the invention have all the functional and physiological properties of native in vivo canine pancreatic islets. In particular, the present inventors have surprisingly and unexpectedly discovered that canine endocrine pancreas-like tissue developed after the transplant of the functional pancreatic islets of the invention under the kidney capsule of an animal, in a reproducible manner. The endocrine pancreas-like tissues thus developed have all the functional and physiological properties of native in vivo canine endocrine pancreas. Thus, the canine pancreatic islets populations of the invention provide an abundant source of stable and functional canine pancreatic islets that can be used for the treatment of canine pancreatic disorders, such as diabetes. Thus, said canine pancreatic islet populations, obtainable by the above-described method, are particularly useful for preventing or treating a canine pancreatic disorder.

In one embodiment, said canine pancreatic islet populations are used for the establishment of master banks useful for cell therapy of canine pancreatic disorders, preferably canine diabetes. Thus, in one aspect, the present invention relates to master cell banks obtained from the canine pancreatic islets of the invention.

Said canine pancreatic islet populations are also particularly useful for testing and screening candidate medicaments for treating canine pancreatic disorders, in vivo after graft in non-human animals, such as mice or rats, or in vitro.

### Methods for Preparing Transduced Canine Pancreatic Islets, Transduced Canine Pancreatic Beta Cells or Canine Beta Cell Tumours

The present inventors have surprisingly discovered that the canine pancreatic islets, obtained by the method described above can be transduced with specific genes.

The present inventors have also surprisingly discovered that, by using a sub-grafting method with the canine pancreatic islets that have been transduced with one or more immortalising gene(s), insulinoma-structures were formed. These insulinoma-structures contain canine functional transduced pancreatic islets, whose sub-grafting results in a specific enrichment in functional transduced pancreatic islets and/or in beta cells, ultimately leading to the production of homogenous, stable and functional transduced canine islet populations or transduced beta cell populations which can be further amplified to clinical and commercial scale. By repeating these enrichment and amplification steps, the inventors were able to obtain repeatedly functional canine islet populations which are capable of stably producing canine insulin and can be amplified for testing, diagnosis or therapeutic use.

Therefore, the present invention is directed in one aspect to a method for preparing transduced canine pancreatic islets, transduced canine pancreatic beta cells or canine beta cell tumours comprising the step of:
a) transducing and co-transducing the canine pancreatic islets of the invention with i) a lentiviral vector expressing SV40 Large T antigen under the control of the insulin promoter, or ii) with a lentiviral vector expressing SV40 Large T antigen under the control of the insulin promoter and a lentiviral vector expressing hTert under the control of the insulin promoter, or iii) a lentiviral vector expressing both SV40 Large T antigen and hTert under the control of the insulin promoter.

In a first embodiment, said method further comprises the step of:
b) collecting the canine pancreatic islets obtained at step a) to form a homogenous transduced canine islet population and optionally growing said population in vitro in an appropriate culture medium.

In a second embodiment, said method further comprises the step of:
b) collecting the canine pancreatic islets obtained at step a) and dissociating the cells thereof, to form a homogenous transduced canine pancreatic beta cell population and growing said transduced canine beta cell population in vitro to establish a canine functional beta cell line.

In a third embodiment, said method further comprises the steps of:
b) dissociating the transduced pancreatic beta cells from the transduced canine pancreatic islets of step a), preferably wherein said dissociation comprises a digestion; and
c) harvesting the pancreatic beta cells contained in the dissociated islets of step b), preferably by centrifugation.

In another embodiment, said method further comprises the steps of:
b) introducing the transduced pancreatic islets obtained in a) into the kidney capsule of a first severe combined immunodeficiency (scid) non-human animal;
c) allowing the transduced pancreatic islets to develop insulinoma-like structures, wherein the canine pancreas cells in insulinoma-like structures have differentiated to insulin-producing pancreatic islets and/or beta cells;
d) micro-dissecting the insulinoma-like structures obtained in step c), and dissociating the islets and/or cells thereof;
e) sub-transplanting the islets and/or cells obtained in step d) into the kidney capsule of a second scid non-human animal;
f) allowing the sub-transplanted islets and/or cells in step e) to develop and regenerate newly developed insulinoma-like structures, wherein said newly developed insulinoma-like structures are enriched in insulin-producing pancreatic islets and/or beta cells;
g) micro-dissecting the insulinoma-like structures obtained in step f), and dissociating and collecting the islets and/or cells thereof;
h) optionally, sub-transplanting the islets and/or cells obtained in step g) into the kidney capsule of a third non-human scid animal, hence allowing further enrichment and amplification of insulin-producing pancreatic islets and/or beta cells; and
i) optionally repeating step e), f) and g) until the appropriate amount of insulin-producing transduced pancreatic islets, of insulin-producing transduced pancreatic beta cells or of canine beta cell tumours is obtained.

By “insulin promoter”, it is herein referred to the genomic region containing the regulatory nucleic acid sequences involved in the regulation of the insulin gene expression. In a preferred embodiment, the insulin promoter used in the invention is a murine insulin promoter. Preferably, insulin promoter used in the invention is the rat insulin promoter. Even more preferably, said rat insulin promoter is the promoter described in Castaing et al., 2005.

Transduction of the immature canine pancreas islets obtained from the dissociation of the pancreatic tissues with lentiviral vectors is carried out according to the methods known to the person of skills in the art (see e.g. Russ et al., 2008 and Khalfallah et al., 2009, and references therein). Lentiviral vectors are vectors derived from a lentivirus such as HIV1. They are able to transduce non-dividing as well as dividing cells and sustain expression of heterologous nucleic acid sequences in several target tissues in vivo, including brain, liver, muscle, and hematopoietic stem cells. A great number of lentiviral vectors are already known to the person of skills in the art; any one of these vectors can be used in the context of the present invention, provided that they express at least the SV40 Large T antigen and/or hTERT, under the control of the insulin promoter. The person of skills in the art is directed to Russ et al., 2008 and Khalfallah et al., 2009 where examples of such lentiviral vectors have been described.

It may be advantageous to de-immortalize the immature canine pancreatic islets or cells obtained by the method described above. For example, if administration of the said islets or cells to a subject is contemplated, it may be safer to remove the oncogenes carried by the vectors. Lentiviral vectors can thus be constructed to allow reversible or conditional immortalization, so that at least one Lox P site may be introduced. More preferably, the vectors according to the invention are constructed so that the SV40 Large T and/or the hTERT transgenes are located within two Lox P site. Said transgenes are removed by expressing the Cre recombinase in the beta cells. For example, the islets obtainable by the above method are transduced by a vector or plasmid expressing a Cre recombinase and reversion occurs. Of course, the skilled in the art may choose to use the FRT/FLP system to remove said transgenes. Methods for reverting immortalized cells are described in WO 01/38548.

In a particular embodiment, the lentiviral vector expressing SV40 Large T and the lentiviral vector expressing hTERT further comprise a LoxP or a FRT site, provided that site specific recombination sites are different in both vectors.

A negative selection step can also be performed after the action of the Cre or FLP recombinase. This further step allows selecting only the islets or cells in which the immortalization genes SV40 Large T and hTERT, as well as the antibiotic resistance gene, have been removed. These islets or cells can be frozen, stored and optionally encapsulated, until they are transplanted into the canine animals in need thereof, such as diabetic canine animals.

The negative selection marker gene can be, for example, the HSV-TK gene and the selective agent acyclovir-ganciclovir. Or the negative selection markers are the hypoxanthine phosphoribosyl transferase (HPRT) gene and the guanine-phosphoribosyl-transferase (Gpt) gene and the selective agent is the 6-thioguanine. Or the negative selection marker is the cytosine deaminase gene and the selective agent is the 5-fluoro-cytosine. Thus, in a preferred embodiment, the said negative marker gene is selected from the group constituted by the HSV-TK gene, the hypoxanthine phosphoribosyl transferase (HPRT) gene, the guanine-phosphoribosyl-transferase (Gpt) gene, and the cytosine deaminase gene. Other examples of negative selection marker proteins are the viral and bacterial toxins such as the diphteric toxin A (DTA). These negative selection genes and agents and their use are well known to the person of skills in the art and need not be further detailed here.

The transduced islets are then introduced into at least one kidney capsule of scid animals as defined above.

Optionally, the islets are further transduced at step a) with another lentiviral vector expressing an antibiotic resistance gene under the control of the insulin promoter. The antibiotic resistance gene is selected in the group consisting of hygromycin resistance gene, neomycin resistance genes, tetracycline resistance gene, ampicillin resistance gene, kanamycin resistance gene, phleomycin resistance gene, bleomycin resistance gene, geneticin resistance gene, carbenicillin resistance gene, chloramphenicol resistance gene, puromycin resistance gene, blasticidin-S-deaminase gene. In a preferred embodiment, said antibiotic resistance gene is a neomycin resistance gene. In this case, the selective agent is G418.

A method for obtaining human pancreatic cells is disclosed in Ravassard et al. (2011) and WO 2008/102000. However, this method does not allow obtaining and identifying mice carrying canine insulinoma and canine pancreatic islets. These publications contain no information regarding dogs and the development of canine pancreas, notably regarding the apparition of insulin-producing cells. Moreover, whereas expression of human insulin confers hypoglycaemia in scid mice, it is not the case with canine insulin. It is therefore not possible to screen scid mice having developed functional canine insulinomas by assaying their glycaemia. Importantly, the inventors were the first to show that canine insulinoma can be detected in the transplanted mice by assaying canine-specific insulin in the mice, allowing for selection of the successfully transplanted mice (PCT/EP2017/061401). Thus, in one embodiment, non-human animals having developed insulinoma-like structures having differentiated to insulin-producing pancreatic islets are selected by measuring the canine-specific insulin level in the non-human animals.

Methods for measuring and/or determining the level of canine-specific insulin are generally known to those skilled in the art and has routinely relied on methods developed to measure human insulin. Methods for measuring and/or determining the level of canine insulin include, for example mass spectrometry, biochemical tests, including immunological tests such as, for example, traditional immunological detection tests (enzyme-linked immunosorbent assay or ELISAs and ELISPOT assays), or such as, for example, immunological tests employing techniques involving transfer of proteins on a support, such as the slot blot (also called dot blot) or the western blot. It is possible, for example, to employ protein microarrays, antibody microarrays or tissue microarrays coupled with immunohistochemistry. Among other techniques that can be used are BRET or FRET techniques, methods of microscopy or histochemistry, including in particular methods of confocal microscopy and electron microscopy, methods based on the use of one or more excitation wavelengths and a suitable optical method, such as an electrochemical method (voltammetry and amperometry), atomic force microscopy, and methods of radio frequency, such as multipolar resonance spectroscopy, confocal and non-confocal, detection of fluorescence, luminescence, chemiluminescence, absorbance, reflectance, transmittance, and birefringence or refraction index (for example, by surface plasmon resonance, by ellipsometry, by a resonant mirror method, etc.), flow cytometry, by radioisotope or magnetic resonance imaging, analysis by polyacrylamide gel electrophoresis (SDS-PAGE); by HPLC-mass spectrophotometry, by liquid chromatography/mass spectrophotometry/mass spectrometry (LC-MS/MS). All these techniques are well-known to the skilled person and it is not necessary to detail them herein.

Thus, in one embodiment, the method of the invention further comprises a step of measuring the level of canine-specific insulin prior to step d) in order to select non-human animals having developed insulinoma-like structures having differentiated to insulin-producing pancreatic islets. In one embodiment, the level of canine-specific insulin is measured using a canine-specific insulin antibody. Such antibodies are commercially available. Advantageously, the canine-specific insulin antibody is comprised in a kit. Preferably, the level of canine-specific insulin is measured by ELISA. Advantageously the level of canine-specific insulin is measured by ELISA using a canine-specific ELISA kit comprising a canine-specific insulin antibody. Canine-specific ELISA kits may further include antibodies, calibrators, buffer, and analytic range optimized for canine insulin.

The above-defined method may include collecting the transduced canine functional pancreatic islets obtained at step g), to form a homogenous transduced islet population. The islet population can further be grown in vitro to establish a homogenous and functional canine transduced pancreatic islet population.

The transduced canine functional pancreatic islets obtained at step g) may also be dissociated to collect transduced beta cells contained in the islets. Said transduced canine beta cells can further be grown in vitro to establish a homogenous and functional canine beta cell line. Thus, in one embodiment, the above method comprises the further step of micro-dissecting the insulinoma-like structures obtained, dissociating the transduced pancreatic islets, preferably wherein said dissociation comprises a digestion, and collecting the islets and/or cells thereof, for example by centrifugation.

The above method to prepare transduced canine functional islets, transduced beta cells or beta cell tumours is particularly useful for preventing or treating a canine pancreatic disorder. The above method to prepare transduced canine functional islets is also particularly useful for testing and screening candidate medicaments for treating canine pancreatic disorders, either in vitro or in vivo, after graft in non-human animals, such as mice or rats. Said method is also useful for in vitro diagnosis of canine diabetes.

In this regard, and in one embodiment, the above method can be put to practice to prepare large amount of transduced canine functional pancreatic islets or beta cells for therapeutic purposes, for testing and screening purposes as well as for in vitro diagnosis of canine diabetes allowing classification of diabetic animals in type 1 diabetes or other types of diabetes. With the above method, large amount of functional transduced canine pancreatic islets or transduced beta cells can be efficiently obtained in vitro. Additionally, steps e), f) and g) can be repeated as many times as necessary to obtain large amount of insulinoma-like structure, functional canine pancreatic islets and functional transduced beta cells. These islets and/or cells may further be amplified in culture in vitro ad infinitum. In still another embodiment, the method for preparing transduced canine pancreatic islets or beta cells as described above is directed to the establishment of master banks for cell therapy of canine pancreatic disorders. Thus, in one aspect, the present invention encompasses a method for the establishment of master cell banks for cell therapy of diabetes, comprising the step of obtaining transduced canine pancreatic islets or canine beta cells by the method described above.

Therefore, the invention is aimed at transduced canine or canine pancreatic islets, transduced canine beta cells, or beta cell tumours obtainable by the above-described method. These transduced canine pancreatic islets, transduced canine beta cells or beta cell tumours display at least one of the following features:
expression of canine-specific insulin and
transcriptional factor Pdx1 positive.

Advantageously, said transduced canine pancreatic islets, canine beta cells or beta cell tumours further display at least one of the following features:
SV40 Large T positive
Carboxypeptidase-A negative
transcription factor MafA positive
proconvertase Pcsk1 positive
expression of Glucose transporter Glut2
expression of Kcnj11 and Abcc8 coding for subunits of the potassium channel
expression of zinc transporter Znt8 (Slc30a8).

Transduced canine pancreatic islets, transduced canine beta cells or beta cell tumours as defined above are also positive to reaction with anti-insulin, anti-GAD and/or anti-IA2 antibodies and can be maintained and grown in culture in a medium free of serum and on Matrigel or on fibronectin coated wells. Indeed, the inventors were the first to show that the transduced canine pancreatic islets, transduced canine beta cells or beta cell tumours, grown and maintained in such medium, are capable of stably, efficiently and homogenously producing canine insulin. Thus, the invention also contemplates a cell culture comprising the above-described canine pancreatic islets or transduced canine beta cells in culture in a medium free of serum comprising Matrigel or fibronectin. This cell culture allows to expand and to establish immortalized canine pancreatic islets populations and immortalized canine beta cell lines.

Moreover, the transduced islet populations or transduced beta cell lines obtainable by the above-described method may be de-immortalized, so that they can be used for example for preventing or treating a canine pancreatic disorder, as well as for testing and screening purposes or for in vitro diagnosis of canine diabetes allowing classification of diabetic animals in type 1 diabetes or other types of diabetes.

In still another embodiment, the method for preparing transduced canine pancreatic islets, transduced canine beta cells or beta cell tumours as described above is directed to the establishment of master banks for cell therapy of canine diabetes. Here, said method further includes de-immortalizing the islets or the cells. Said de-immortalization of the cells includes a step of removing the SV40 Large T and the hTERT transgene from the lentiviral vectors. Preferably the transgenes are excised by site-specific recombination with a site-specific recombinase such as Cre or FLP, as described above.

The invention also concerns the use of said transduced canine pancreatic islets, canine beta cells or beta cell tumours as described above for cell therapy of canine pancreatic disorder. Here, said method may further includes de-immortalizing the islets or the cells.

### Methods of Prevention and Treatment and Veterinary Composition

The present inventors have shown that the canine pancreatic islets produced by the methods described above (native or transduced) can be successfully grafted in animals and developed fully functional endocrine pancreas-like tissue. The canine pancreatic islets produced by the methods described above can be used to regenerate canine endocrine pancreas functions in an individual animal. This opens considerable perspective towards veterinary use of such pancreatic islets in the treatment of canine pancreatic disorders, such as diabetes.

Accordingly, the present invention also provides a method of regenerating canine endocrine pancreas functions in an individual animal afflicted with a canine pancreatic disorder, such as canine diabetes. Indeed, the canine pancreatic islets of the invention have all the functional and physiological properties of native in vivo canine pancreatic islets. In particular, they are capable of producing canine insulin and glucagon and are capable of responding to glucose stimulation. The present invention also relates to the canine pancreatic islets of the invention for use for regenerating canine endocrine pancreas functions in an individual animal afflicted with a canine pancreatic disorder, such as canine diabetes.

In one embodiment, the method of or the use for regenerating canine endocrine pancreas function in an individual animal afflicted with a canine pancreatic disorder comprises a step of administrating an effective amount of the canine functional pancreatic islets as defined above, the transduced canine functional pancreatic islets as defined above, or the de-immortalised functional pancreatic islets as defined above, into said animal. In a preferred embodiment, said islets are transplanted within said animal. In another preferred embodiment, said method of regenerating pancreas function comprises a prior step of obtaining the said canine pancreatic islets by the methods described above.

The invention also relates to a pharmaceutical composition comprising a pharmaceutical acceptable carrier and an effective amount of the canine functional pancreatic islets as defined above, the transduced canine functional pancreatic islets as defined above, or the de-immortalised functional pancreatic islets as defined above, said cells being optionally encapsulated.

In one aspect, the invention concerns a veterinary composition comprising a pharmaceutically acceptable carrier and an effective amount of the canine pancreatic islets as described above, the transduced canine functional pancreatic islets as defined above, or the de-immortalised functional pancreatic islets as defined above. In one embodiment, the canine pancreatic islets in said pharmaceutical or said veterinary composition are encapsulated in a protective device, preferably a protective capsule.

An “effective amount” is an amount sufficient to effect beneficial or desired clinical results. An effective amount, for example from 10⁵ to 10⁹ cells, can be administered in one or more applications, although it is preferable that one administration will suffice. For purposes of this invention, an effective amount of pancreatic islets is an amount that is sufficient to produce differentiated pancreatic islets which are able to restore one or more of the functions of the pancreas. It is contemplated that a restoration can occur quickly by the introduction of relatively large numbers of pancreatic islets, for example greater than 10⁵ islets. In addition, it is also contemplated that when fewer pancreatic islets are introduced, function will be restored when the pancreas islets are allowed to proliferate in vivo. Thus, an “effective amount” of pancreatic islets can be obtained by allowing as few as one pancreas islet sufficient time to regenerate all or part of a pancreas. Preferably, an effective amount administered to the individual is greater than about 10¹ pancreatic islets, preferably between about 10² and about 10¹⁵ pancreatic islets and even more preferably, between about 10³ and about 10¹² pancreatic islets. In terms of treatment, an “effective amount” of pancreatic islets is the amount which is able to prevent, ameliorate, palliate, stabilize, reverse, slow or delay the progression of pancreas disease, such as diabetics.

As used herein, “pharmaceutically acceptable carrier” includes any and all solvents, buffers, salt solutions, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of media and agents for pharmaceutically active substances is well known in the art. A typical pharmaceutical composition for intravenous infusion could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17th ed., Mack Publishing Company, Easton, Pa. (1985), and the 18^{th} and 19^{th} editions thereof, which are incorporated herein by reference.

The canine pancreatic islets of the invention, including the functional canine pancreatic islets as defined above, the transduced canine functional pancreatic islets as defined above, or the de-immortalised functional pancreatic islets as defined above, can be useful for regenerating pancreatic functions. Said islets can also be administered to an animal suffering from a pancreatic disorder in order to treat said disorder. Thus, the present invention concerns the canine pancreatic islets obtained by the method of the invention, as described above, the functional canine pancreatic islets as defined above, or the transduced canine functional pancreatic islets as defined above, for use in preventing or treating a canine pancreatic disorder. In one embodiment, said canine pancreatic islets are encapsulated in a protective device, preferably a protective capsule. According to a preferred embodiment, the canine pancreatic tissue used in the method of the invention for obtaining said canine pancreatic islets is obtained the animal in need of a treatment. The present invention also concerns the pharmaceutical composition or veterinary composition described above, for use in preventing or treating a canine pancreatic disorder.

The present invention also concerns a method for treating a canine pancreatic disorder with the functional canine pancreatic islets obtained by the method of the invention as defined above, the transduced canine functional pancreatic islets as defined above, or the de-immortalised functional pancreatic islets as defined above, comprising the administration of said canine pancreatic islets, or administration of the pharmaceutical composition or veterinary composition thereof, to an animal in need thereof. In one embodiment, said canine pancreatic islets are encapsulated in a protective device, preferably a protective capsule. According to a preferred embodiment, the treatment method of the invention comprises a prior step of obtaining the said canine pancreatic islets from a canine pancreatic tissue. In a further preferred embodiment, the canine pancreatic tissue is obtained from said animal in need of a treatment.

The present invention also concerns a method for preventing a canine pancreatic disorder with canine pancreatic islets obtained by the method of the invention, as defined above, the transduced canine functional pancreatic islets as defined above, or the de-immortalised functional pancreatic islets as defined above, comprising the administration of said canine pancreatic islets, or the administration of said pharmaceutical composition or said veterinary composition comprising said pancreatic islets, to an animal in need thereof. An animal in need thereof may be an animal at risk of developing a pancreatic disorder. According to a preferred embodiment, the prevention method of the invention comprises a prior step of obtaining the said canine pancreatic islets from a canine pancreatic tissue. In a further preferred embodiment, the canine pancreatic tissue is obtained from said animal in need of a treatment.

It is another aspect of the present invention to provide canine pancreatic islets of the invention as a medicament. More precisely, the present invention relates to the use of canine pancreatic islets of the invention, including the functional canine pancreatic islets obtained by the method of the invention, as defined above, the transduced canine functional pancreatic islets as defined above, or the de-immortalised functional pancreatic islets as defined above, or of the pharmaceutical composition or the veterinary composition as described above, for preparing a medicament to prevent or treat a canine pancreatic disorder.

Methods of introducing pancreatic islets into canine animals are well known to those of skills in the art. In one embodiment, with respects to any of the aspects of the invention described above, said canine pancreatic islets or said veterinary or pharmaceutical composition are (is) transplanted in the pancreas, the liver, a muscle, a subcutaneous tissue, the renal subcapsule, the peritoneal cavity of said animal. The canine pancreatic islets of the invention or compositions thereof can also be introduced into any other sites, including but not limited to the abdominal cavity, the kidney, the liver, the portal vein or the spleen. Preferably, said islets or compositions are deposited in the pancreas of the animal.

In another embodiment, said canine pancreatic islets or said veterinary or pharmaceutical composition are (is) administered by injection in said animal, preferably by intraperitoneal, subcutaneous, intravenous or intraportal injection, with a specific preference for intraperitoneal injection. Single, multiple, continuous or intermittent administration can be done.

A “canine pancreatic disorder” according to the invention includes diabetes, hypoglycaemia, or any pathology associated with a dysfunction of the digestive enzymes. Preferably, a canine pancreatic disorder is insulin-dependent diabetes (T1 D).

As used herein, “preventing a canine pancreatic disorder” means reducing the risk of developing a canine pancreatic disorder.

By “diabetes”, it is herein referred to a chronic, often debilitating and sometimes fatal disease, in which the body either cannot produce insulin or cannot properly use the insulin it produces. A canine type 1 diabetes according to the invention is a diabetes resulting from autoimmune destruction of beta cells. As used herein, “other types of diabetes in dogs” or “other types of canine diabetes” refer to canine diabetes which are not of the type 1.

### Methods of Screening and of Diagnosing

The above described methods to prepare canine functional pancreatic islets, transduced canine functional pancreatic islets as defined above, transduced beta cells or de-immortalised functional pancreatic islets as defined above are particularly useful for testing and screening candidate medicaments for treating canine diabetes in vivo after graft in non-human animals, such as mice or rats, or in vitro. Indeed, the canine pancreatic islets of the invention have all the functional and physiological properties of native in vivo canine pancreatic islets. In particular, they are capable of producing canine insulin and glucagon. They are capable of responding to glucose stimulation.

Specifically, the invention relates to a method for testing and screening candidate medicaments for treating canine diabetes, said method comprising the step of administering a candidate medicament to a non-human animal grafted with the canine pancreatic islets of the invention. In a more specific embodiment, the method comprises prior steps of obtaining said pancreatic islets according to the methods described above, and grafting said islets into the said non-human animal. Said non-human animal is preferably a scid non-human animal, as described above. The invention also relates to a method for testing and screening in vitro candidate medicaments for treating canine diabetes, said method comprising the step of administering a candidate medicament to the culture of functional canine pancreatic islets of the invention as described above, or to the culture of transduced canine functional pancreatic islets as defined above, or the de-immortalised functional pancreatic islets as defined above or a culture of transduced beta cell lines as described above.

The present invention also relates to a method of in vitro diagnosis of canine pancreatic disorders, preferably canine diabetes. Cross section of insulinoma-like structures, of canine pancreatic islets or canine beta cells obtained by the methods described above or protein extract from these insulinoma, islets or cells can be bound or adsorbed to a solid support (for example polylysine coated plates) and reacted with the plasma serum of canine animals. After incubation, the serum is washed out and the presence or absence of autoantibodies against different surface antigens specific to autoimmunity associated with diabetes is revealed (for example by means of labelled anti-canine Ig).

Thus, in one embodiment, the invention relates to a method of in vitro diagnosis of canine diabetes comprising linking or adsorbing insulinoma-like structures as described above, functional canine pancreatic islets as defined above, transduced canine functional pancreatic islets as defined above, or de-immortalised functional pancreatic islets as defined above, or transduced beta cells as defined above, or protein extracted from said insulinoma or islets or cells, to a solid support and reacting with the plasma serum of animals, detecting the presence or absence of auto-antibodies against different surface antigen specific to canine diabetes type 1 or other types of diabetes, such as Islet Cells Antibodies (ICA), selected for example from Insulin auto-antibodies (IAA) and glutamic acid decarboxylase antibodies (GADA).

Preferably, sera from diabetic animal and control animal are added on said tissue sections of said insulinoma-like structures or canine pancreatic islets or canine beta cells, and incubated with a labelled anti-canine IgG, such as a fluorescent labelled conjugated anti-canine IgG, in order to reveal the presence or absence of auto-antibodies associated with canine diabetes in the sera of said patient animal. In this embodiment, the presence of auto-antibodies is indicative of canine diabetes.

The presence or absence of auto-antibodies associated with canine diabetes in the sera of said diabetic animal can also be detected by a western blot of a protein extract of said insulinoma-like structures or canine pancreatic islets or beta cells. In this case, the presence or absence of auto-antibodies associated with canine diabetes in the sera of said diabetic animal is detected with labelled anti canine IgG, such as HRP conjugated anti canine IgG. Alternatively, the presence or absence of auto-antibodies associated with canine diabetes in the sera of said diabetic animal is detected by an ELISA test in which the wells plates are coated with a protein extract of said canine pancreatic islets, said canine beta cell tumours or said canine pancreatic beta cells. According to this embodiment, said protein extract is incubated with sera from diabetic animal and control animal, and the presence or absence of auto-antibodies associated with canine diabetes in the sera of said diabetic animal is detected with labelled anti canine IgG, such as HRP conjugated anti canine IgG.

In another aspect, a method of in vitro diagnosis of canine diabetes comprises reacting section of insulinoma-like structures or canine pancreatic islets or beta cells obtainable by the methods depicted above, or protein extracted therefrom, with the plasma serum of animals, detecting the presence or absence of autoantibodies against different surface antigen specific to canine type 1 diabetes or other types of canine diabetes, such as Islet Cells Antibodies (ICA), or more specific antibodies recently identified like antibodies against Insulin autoantibodies (IAA) and glutamic acid decarboxylase antibodies (GADA) or IA-2 antibodies (IA2A) or specific unknown antibodies. The identification of known or new antibodies can be performed by immunoblot or dot-blot for example.

This aspect of the invention provides for the first time a kit that can be prepared at a commercial scale for diagnosing canine diabetes and for classification of diabetes type. More particularly, this kit can be used to detect specific canine autoantibodies such as Islet Cells Antibodies (ICA) selected from Insulin autoantibodies (IAA) and glutamic acid decarboxylase antibodies (GADA). Indeed, these antigens are expressed at the surface of the insulinoma-like structures or canine pancreatic islets or canine beta cells obtainable according to the above method. Thus, embraced herein is a diagnostic kit for canine diabetes, said kit comprising canine pancreatic islets (transduced or not), canine beta cell tumours or canine functional pancreatic beta cells obtainable by the above method, or proteins extract there from, optionally bond or adsorbed to a solid support.

In another embodiment, the functional canine pancreatic islets as defined above, the transduced functional canine pancreatic islets as defined above, or the de-immortalised functional pancreatic islets as defined above, are grown in vitro and canine pancreatic islet populations are established for screening compounds capable of modulating insulin secretion. The present invention thus also provides a method for screening compounds capable of modulating insulin secretion, said method comprising the steps of: a) contacting the canine pancreatic islets of the invention with a test compound, and b) detecting insulin secretion and measuring the level of insulin secretion. Insulin secretion can be detected by any of the means known to the person of skills in the art, as detailed in e.g. the experimental examples below, in Ravassard et al, and in WO 2008/102000. According to a preferred embodiment, the screening method of the invention comprises a step of comparing the level of secreted insulin obtained in step b) with at least one control level. Said control level corresponds to the level of insulin produced by a cell line which is known to secrete insulin. Alternatively, said control level corresponds to the level of insulin produced by a cell line which is known not to produce any insulin. In a further preferred embodiment, the secreted insulin level of step b) is compared with two control levels, one corresponding to the level of insulin produced by a cell line which is known to secrete insulin and the other one corresponds to the level of insulin produced by a cell line which is known not to secrete insulin. In yet another preferred embodiment, the screening method of the invention comprises a prior step of obtaining the canine pancreatic islet population according to the methods for preparing canine pancreatic islets described above.

### FIGURE LEGENDS

FIG. 1: Pancreatic neo-islets after three days of culture of immature pancreatic islets obtained from of a dog foetus at 53pc (E-53). X20

FIG. 2: Pancreatic neo-islets after 7 days of culture of immature pancreatic islets obtained from of a dog foetus at 53pc (E-53). X10

FIG. 3: The dog pancreatic neo-islets are functional and produce dog insulin and glucagon

A) Immunostaining of endocrine markers (insulin (light grey) and glucagon (white)) of pseudo-pancreatic islets after six days of culture of immature pancreatic islets obtained from of a dog foetus at 53pc (E-53). X20.

B) Immunostaining of endocrine markers (insulin (light grey) and glucagon (white)) of pseudo-pancreatic islets after eight days of culture of immature pancreatic islets obtained from of a dog foetus at 53pc (E-53). X10.

FIG. 4: Endocrine pancreas-like tissue obtained after grafting of non-transduced canine pancreatic neo-islets in a scid mice: the pancreatic neo-islets are functional and produce dog insulin and glucagon

Immunostaining of endocrine markers (insulin (light grey) and glucagon (white)) of Endocrine pancreas-like tissue obtained after grafting non-transduced pancreatic islets in scid mice, 2 months post-graft. X20

FIG. 5: Large T positive neo-islets obtained after grafting of Large T transduced canine pancreatic neo-islets in a scid mice: the transduced pancreatic neo-islets are functional and produce dog insulin and glucagon

Immunostaining of endocrine markers (insulin (light grey) and glucagon (white)), of Large T positive neo-islets obtained after grafting Large T transduced-pancreatic neo-islets in scid mice, 2 months post-graft. X10.

FIG. 6: Large T positive neo-islets obtained after grafting of Large T transduced canine pancreatic neo-islets in a scid mice: the transduced pancreatic neo-islets and produce dog insulin and regulate blood glucose concentration

Immunostaining of insulin (light grey) of Large T positive neo-islets, obtained after grafting Large T transduced-pancreatic neo-islets in scid mice.

FIG. 7: Beta cell lines obtanined from Large T positive neo-islets produce dog insulin

Immunostaining of endocrine marker insulin (light grey) of beta cells obtained from a Large T positive neo-islets obtained after grafting Large T transduced-pancreatic neo-islets in scid mice.

### EXAMPLES

### A) Material and Methods

A.1. Materials

HBSS (Hanks' Balanced Salt Solution) is supplemented with 5.6 mM glucose; 0.2 mg/mL BSA fat acid free and 1% penicillin-streptomycin.

The culture medium is made with a base of RPMI 1640 medium already containing 11 mM glucose and 25 mM Hepes and supplemented with 10% FCS and 1% penicillin-streptomycin.

A.2. Source of canine pancreatic tissue and collection procedure

Pancreases were obtained from Beagle dogs, a strain raised in the housing facilities of Maison-Alfort Veterinary School, at foetal stage 53 days pc (post conception, E-53). All foetal samples were obtained by elective caesarean section. The foetal age was determined according to the ovulation identified by the plasma progesterone surge

All the procedures involving animals were approved by the Ethic Committee of Maison-Alfort Veterinary School.

A.3. Generation of the Canine Pancreatic Islets

Immediately after surgery, all pancreases were dissected and minced into 1 mm square pieces in supplemented HBSS. The pancreas pieces were digested with collagenase A at 6 mg/mL at 37° C. for 4-6 min. The digestion was stopped by dilution with cold supplemented HBSS. The digested pieces were washed twice.

The digested pancreas pieces were collected and incubated in the culture medium defined in A.1.

A.4. Immunohistochemistry

The pseudo-islets were fixed in 4% PFA (paraformaldehyde) and embedded in gelatine-sucrose. Sections were cut them with cryostat.

Sections were stained with a guinea pig anti-insulin antibody (1/500; A0564, Dako-Cytomation) and rabbit anti-glucagon (1/1000; 20076-Immuno, Euromedex). The secondary antibodies were fluorescein Texas red anti-guinea pig antibody (1/2000; 706-076-148, Jackson and anti-rabbit antibody (1/200; 711-096-152, Jackson Immunodetect Laboratories, Beckman Coulter). Cell nuclei were stained with Hoechst or DAPI. Digital images were captured using an Axio Scan Z1 (Zeiss).

A.5. DNA Constructs and Recombinant Lentiviral Productions

The lentiviral vectors, pTRIP ΔU3.RIP405-SV40LT loxP and pTRIP ΔU3.RIP405-hTERT loxP, have been constructed by adding a loxP site in the 3′LTR region of the pTrip ΔU3.RIP405-SV40LT/hTERT previously described (Ravassard et al, 2009). Both pTRIP ΔU3 vectors were digested by KpnI and PacI to remove the 3′LTR region. The 3′LTRloxP region of the SIN-RP-LTcDNA-WHV-U3loxP (provided by Bernard Thorens) was amplify by PCR and next digested by KpnI and PacI and then ligated into the two linearized pTrip vectors. The Lentiviral vector stocks were produced by transient transfection of 293T cells by encapsidation of the p8.9 plasmid (ΔVprΔVifΔVpuΔNef), pHCMV-G that encoded the VSV glycoprotein-G and the pTRIP ΔU3 recombinant vector, as previously described (Zufferey et al., 1997). The supernatants were treated with DNAse I (Roche Diagnostic) prior to their ultracentrifugation, and the resultant pellets were re-suspended in PBS, aliquoted, and then frozen at −80° C. until use. The amount of p24 capsid protein was quantified by the HIV-1 p24 antigen ELISA (Beckman Coulter). All transductions were normalized relative to p24 capsid protein quantification.

A.6. Gene Transfer

The pseudo-islets to be transduced were incubated with a total amount of lentiviral vectors (pTRIP ΔU3.RIP405-SV40LT loxP) corresponding to 2 μg of p24 capside protein for 1 hour at 37° C. in of DMEM that contained 5.6 mM glucose, 2% bovine serum albumin fraction V (BSA, Roche diagnostics), 50 μM 2-mercaptoethanol, 10 mM nicotinamide (Calbiochem), 5.5 μg/ml transferrin (Sigma-Aldrich), 6.7 ng/ml selenite (Sigma-Aldrich), 100 U/ml penicillin, and 100 μg/ml streptomycin and 10 μg/ml DEAE-dextran (DEAE for Diethylaminoethyl). The transduction reaction was diluted in the culture medium and the transduced islets were kept on culture overnight until transplantation into scid mice.

A.7. Animals and Transplantation into Scid Mice

Male scid mice (Harlan) were maintained in isolators. Diabetes was induced in the scid mice by treating said mice with streptozotocine as described previously (Ravassard et al., 2009; Ravassard et al., 2011). Using a dissecting microscope, islets were implanted under the kidney capsule, as previously described (Ravassard et al., 2011). At different time points after transplantation, the mice were sacrificed, the kidney removed, and the graft dissected. All animal studies and protocols were approved by the Veterinary Inspection Office in compliance with the French legislation under agreement number B75-13-03.

A.8. Assay of Dog-Specific Insulin Levels

The levels of dog-specific insulin were assayed using an ELISA kit commercialized by MERCODIA, following the instructions of the manufacturer.

### B) Production of Functional Canine Pancreatic Neo-Islets

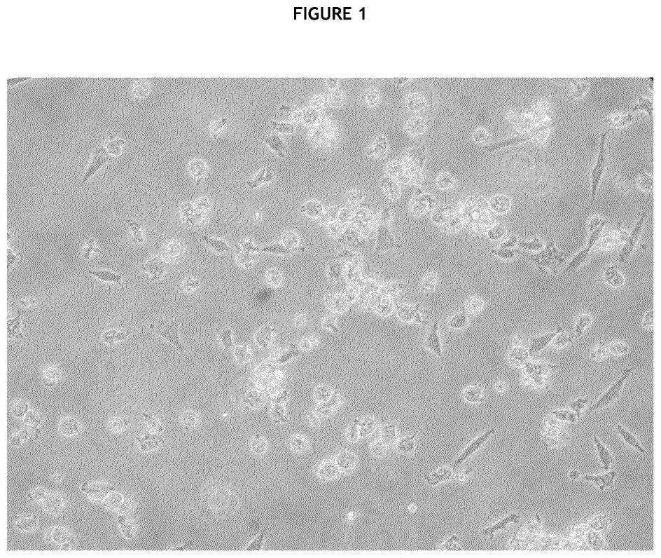

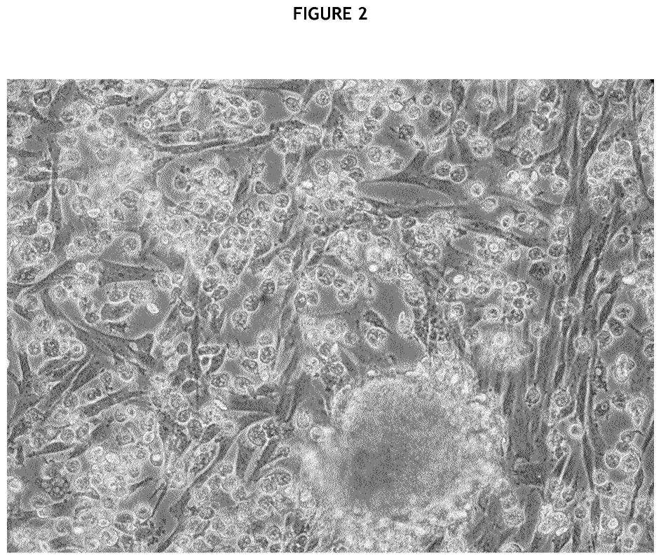

Dog islets were prepared and grown as described in section A.3 above. The evolution of the cultures was monitored by microscopy. A network of fibroblastic type cells begin to form after two days of culture (D+3; FIG. 1). At D+4 pancreatic islet-like structures (pseudo-islets) begin to form. At D+7, spherical pancreatic neo-islet structures are formed (FIG. 2). These results show that the method developed by the inventors allows rapid, efficient and easy de novo generation of dog pancreatic neo-islets.

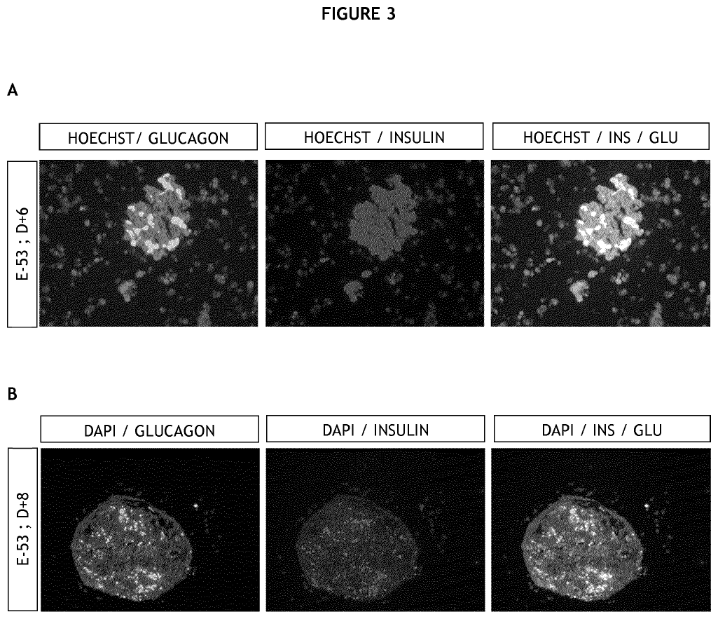

The dog islets were studied by immunohistochemistry. Cells were stained with an anti-insulin antibody (light grey), an anti-glucagon antibody (white) and the nuclei were stained with Hoechst (dark grey; FIG. 3). FIGS. 3 A and B shows that the size of the neo-islets increases with the time of culture and that high levels of insulin and glucagon are detected in all the pseudo-islets. Both insulin and glucagon expressions are detected after more than 21 days of culture (data not shown). Dog-specific insulin secretion in the culture medium was assayed as described in section A.8 above. Insulin was detected in all batches.

These results show that the dog neo-islets are homogeneous and are stably producing insulin and glucagon and are capable of secreting insulin.

Insulin secretion was further assayed upon glucose stimulation. Increasing the glucose concentration in the medium to 15 mM resulted in a 1.5 to 4-fold increase in insulin secretion. Therefore, the dog neo-islets are capable of responding to glucose stimulation.

These data show that the dog islets obtained de novo using the method developed by the inventors are fully functional and stable.

### C) Grafting of Canine Pancreatic Islets

C1. Grafting of Non-Transduced Canine Pancreatic Islets

Dog islets were prepared as described in section A.3 above. The islets were implanted under the kidney capsule of scid mice. The development of endocrine pancreas-like tissue was confirmed by assaying dog-specific insulin in the transplanted mice (as described in EP2017/061401). The grafts were harvested two months after transplantation. The grafts were dissected and fixed in 3.7% formaldehyde prior to their embedding in paraffin.

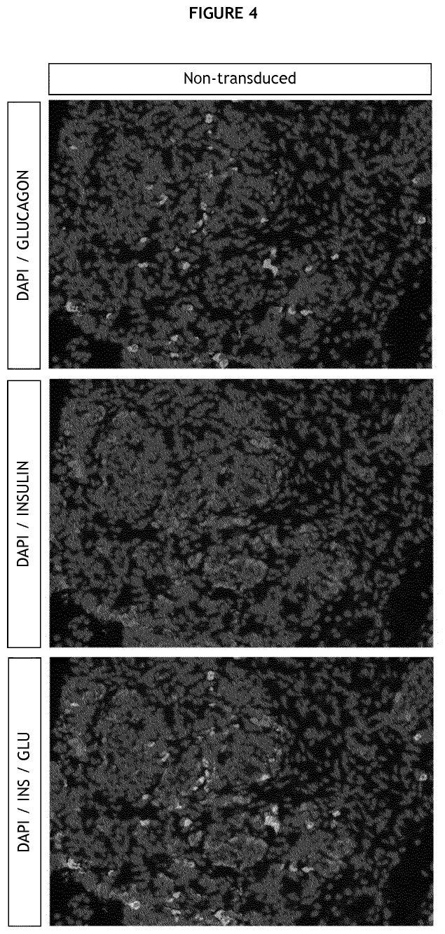

Paraffin-embedded sections were cut and stained with an anti-insulin antibody (light grey), an anti-glucagon antibody (white) and the nuclei were stained with Hoechst (dark grey; FIG. 4), as described in section A.6 above.

FIG. 4 shows that endocrine pancreas-like structures have developed under the kidney capsule of the grafted scid mice. High levels of insulin and glucagon are homogeneously detected in the neo-islets in the structures. These data show that the neo-islets obtained after transplant of non-transduced dog islets are fully functional. These results show that the canine pancreatic islets produced by the method described above can be successfully grafted in animals and develop fully functional endocrine pancreas-like structures. This opens considerable perspective towards veterinary use of such pancreatic islets in the treatment of canine pancreatic disorders, such as diabetes.

Moreover, these result show that pancreatic islets obtained by the method of the invention may be further expanded and maintained in vivo by sub-grafting said islets in scid mice.

C2. Grafting of Large T-Transduced Canine Pancreatic Islets

C2.1. Dog islets were prepared and transduced with Large T expressing vectors as described in sections A.3 and A.6 above. Large T-transduced islets were implanted under the kidney capsule of scid mice as described in section C.1 above.

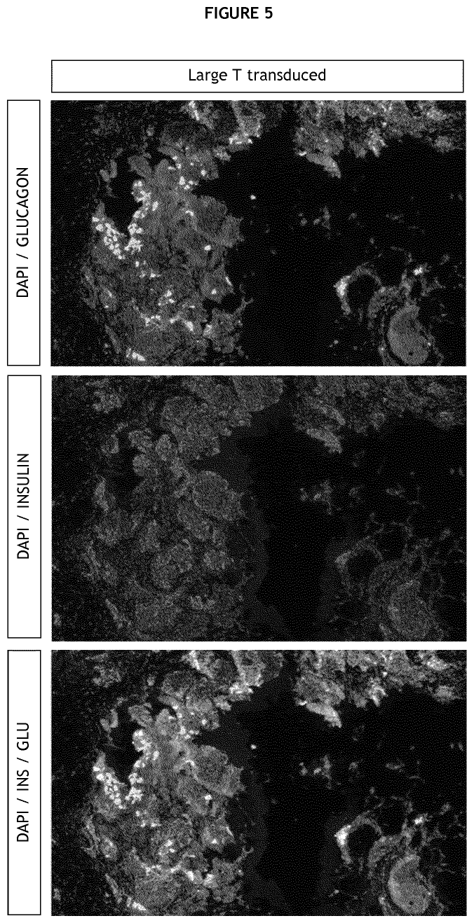

FIG. 5 shows that Large T-positive neo-islets have developed under the kidney capsule of the grafted scid mice. High levels of insulin and glucagon are homogeneously detected in the islets. These data show that the dog pancreatic islets produced by the method described above can be successfully grafted in animals and developed fully functional Large T-positive neo-islets.

C2.2. Dog neo-islets were prepared as described in section A.3 and transduced with lentiviral vectors, pTRIP ΔU3.RIP405-SV40LT loxP described in section A.S. For the transduction, a total amount of lentiviral vector corresponding to 1 μg of p24 capsid protein was used to transduce 10⁶ dog neo-islets in culture. 2 hours after transduction the culture medium is changed. 24 h later the transduced neo-islets were transplanted under the kidney capsule of a scid mice as described in section A.7. 10⁶ to 2×10⁶ transduced pseudo islets were transplanted per mouse. The glucose concentration in the blood of the SCID mice host was assayed.

The data show that a decline in blood glucose concentration from mild to severe hypoglycemia can be observed in the host SCID mice.

In addition, a significant amount of dog insulin was measured in the blood of the host SCID mice.

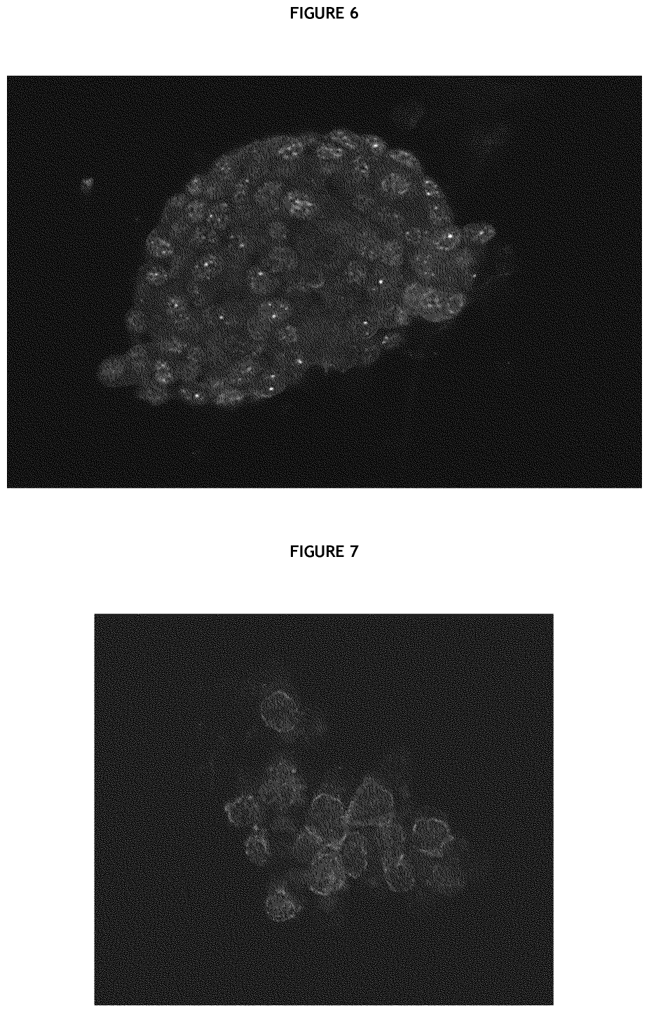

An insulinoma was obtained (FIG. 6). FIG. 6 shows that the cells of the insulinoma were stained for insulin (light grey) and are Large T-positive (white). The Large T oncogene has been transduced in the nucleus of the cells.

The insulinomas were dissociated and cells (beta cells) were collected and expended to obtained a master cell bank of immortalized cells. As shown in FIG. 7, the expended beta cells were positive for insulin, as shown by immune cyto-chemistry (insulin in stained in light grey and nuclei are stained in dark grey). In addition, cells were cultivated and insulin was measured in the milieu. The data show a high concentration of dog insulin secreted by those cells.

This opens considerable perspective towards veterinary use of such pancreatic islets in the treatment of canine pancreatic disorders, such as diabetes.

### REFERENCES

Ahlgren K M, Fall T, Landegren N, Grimelius L, von Euler H, Sundberg K, “Lack of evidence for a role of islet autoimmunity in the aetiology of canine diabetes mellitus”. PLoS One. (2014); 9(8):e105473.
Ausubel F. M. et al, eds., 1987 Current Protocols in Molecular Biology, and periodic updates.
Barbas et al., 2001, Phage Display: A Laboratory Manual.
Bonnet B N. a Egenvall A. “Age patterns of disease and death in insured Swedish dogs, cats and horses”, Department of Population Medicine, University of Guelph, Ontario, Canada. J Comp Pathol. 2010 January; 142 Suppl 1:S33-8.
Bricout-Neveu E, Pechberty S, Reynaud K, Maenhoudt C, Lecomte M J, Ravassard P, and Czernichow P. “Development of the Endocrine Pancreas in the Beagle Dog: From Fetal to Adult Life”. Anat Rec. 2017 Mar. 14. doi: 10.1002/ar.23595.
Castaing, M., Duvillie, B., Quemeneur, E., Basmaciogullari, A., and Scharfmann, R. (2005). “Ex vivo analysis of acinar and endocrine cell development in the human embryonic pancreas”. Dev Dyn. 234, 339-345.
Catchpole B., Ristic J M., Fleeman L M. a Davison L I “Canine diabetes mellitus: can old dogs teach us new tricks?” Diabetologia, 2005; 48:1948-56.
Davison L J, Weenink S M, Christie M R, Herrtage M E, Catchpole B. “Autoantibodies to GAD65 and IA-2 in canine diabetes mellitus”. Veterinary immunology and immunopathology. (2008); 126:83-90.
Davison L I, Herrtage M E. a Catchpole B. “Study of 253 dogs in the United Kingdom with diabetes mellitus” inVet Rec. 2005; 156(15):467-71.
Freshney R. I., ed., 1987. Animal Cell Culture.
Gait M. J., ed., 1984, Oligonucleotide Synthesis.
Gale E. A. M. «Do dogs develop autoimmune diabetes?» Diabetologia (2005) 48: 1945-1947.
Hawkins, K. L., Summers, A., P. Kuhajda, P., Smith, C. A. “Immunocytochemistry of Normal Pancreatic Islets and Spontaneous Islet Cell Tumors in Dogs”. Vet. Pathol. 24: 170-179 (1987).
Justice, D., Cruccioli, N., Roque, C., Galls, J, F., Remaudet, B., Cahard, D. «Etude morphometrique des ilots de Langerhans du pancreas du chien Beagle» Rev Fr Histotechnology 1997, 10:45-49.
Kennedy L J, Davison L J, Barnes A, Short A D, Fretwell N, Jones C A, et al. “Tissue Antigens. Identification of susceptibility and protective major histocompatibility complex haplotypes in canine diabetes mellitus.” (2006); 68(6):467-76.
Khalfallah, O., Ravassard, P., Serguera-Lagache C., Fligny, C., Serre, A., Bayard, E., Faucon-Biguet, N., Mallet, J., Meloni, R., and Nardelli, J. (2009). “Zinc finger protein 191 (ZNF191/Zfp191) is necessary to maintain neural cells as cycling progenitors”. Stem Cells 27:1643-1653.
Kim A, Miller K, Jo J, Kilimnik G, Wojcik P, and Hara M. “Islet architecture: A comparative study”. Islets. 2009; 1(2): 129-136.
Mullis et al, ed., 1994. PCR: The Polymerase Chain Reaction.
Nelson R W and Reusch C E, (2014), “Animal models of disease: classification and etiology of diabetes in dogs and cats”. J Endocrinol. September; 222(3):T1-9. doi: 10.1530/JOE-14-0202.
Niessen S J., Powney S. Guitian J., Niessen A P., Pion P D., Shaw J A. a Church D B. “Evaluation of a quality-of-life tool for dogs with diabetes mellitus” J Vet Med. 2012; 26(4):953-61.
O'Kell A L, Wasserfall C, Catchpole B, Davison L J, Hess R S, Kushner J A, Atkinson M A. “Comparative Pathogenesis of Autoimmune Diabetes in Humans, NOD Mice, and Canines: Has a Valuable Animal Model of Type 1 Diabetes Been Overlooked?” Diabetes. 2017 June; 66(6):1443-1452.
Perbal Bernard V., 1988, A Practical Guide to Molecular Cloning.
Pictet, R. L., Clark, W. R., Williams, R. H., and Rutter, W. J. “An ultrastructural analysis of the developing embryonic pancreas”. Dev. Biol. 1972, 29, 436-467.
Rand J S, Fleeman L M, Farrow H A, Appleton D J, Lederer R. (2004) “Canine and feline diabetes mellitus: nature or nurture?” J Nutr. 2004 August; 134(8 Suppl):20725-20805.
Ravassard P, Emilie Bricout-Neveu, Hazhouz Y, Pechberty S, Mallet J, Czernichow P, Scharfmann R. (2009) “A new strategy to generate functional insulin-producing cell lines by somatic gene transfer into pancreatic progenitors”. PLoS One. 4(3): e4731.
Ravassard P, Hazhouz Y, Pechberty S, Bricout-Neveu E, Armanet M, Czernichow P, Scharfmann R.; (2011) “A genetically engineered human pancreatic B cell line exhibiting glucose-inducible insulin secretion”. J Clin Invest. 2011 September; 121(9):3589-97.
Remington's Pharmaceutical Science, 17^{th} ed., Mack Publishing Company, Easton, Pa. (1985).
Russ, H. A., Bar, Y., Ravassard, P., and Efrat, S. (2008). In vitro proliferation of cells derived from adult human beta-cells revealed by cell-lineage tracing. Diabetes 57:1575-1583.
Sambrook et al, 1989, Molecular Cloning: A Laboratory Manual, second edition.
Scharfmann R, Duvillie B, Stetsyuk V, Attali M, Filhoulaud G, Guillemain G. Beta-cell development: the role of intercellular signals. Diabetes Obes Metab. 2008 November; 10 Suppl 4:195-200.
Shield E J et al, “Extreme Beta-cell deficiency in Pancreata of Dogs with canine Diabetes”, PloS one (2015); 10, 1719.
Steiner D J, Kim A, Miller K, Hara M., “Pancreatic islet plasticity: interspecies comparison of islet architecture and composition”. Islets. 2010; 2(3):135-45.
Tuch B. E. and Madrid J. C., “Development of fetal sheep pancreas after transplantation into athymic mice”. Cell Transplant. 1996 July-August; 5(4):483-9.
Tuch B E, Madrid J C, Summers E, Smith M S. “Production and characterization of fetal sheep pancreatic islet-like cell clusters Cell Transplant”. 1996 July-August; 5(4):491-8.
Woolcott O. O., Bergman R N., Richey J M., Kirkman E L., Harrison L N., lonut V., Lottati M., Zheng D., Hsu I R., Stefanovski D., Kabir M., Kim S P., Catalano K J., Chiu J D., Chow R H., “Simplified Method to Isolate Highly Pure Canine Pancreatic Islets”. Pancreas 2012; 41(1):31-38.
Zufferey, R., Nagy, D., Mandel, R. J., Naldini, L., and Trono, D. (1997). “Multiply attenuated lentiviral vector achieves efficient gene delivery in vivo”. Nat Biotechnol 15, 871-875.

1. A method for producing canine pancreatic islets, comprising the steps of:
a) obtaining canine pancreatic endocrine cells from an immature canine pancreas or a portion thereof; and
b) incubating the endocrine cells of step a) in an appropriate culture medium comprising glucose at a concentration comprised between 4 mM to 30 mM, allowing pancreatic islets to develop.

2. The method according to claim 1, wherein step a) comprises mincing said immature canine pancreas or portion thereof and/or digesting said immature canine pancreas or portion thereof with an appropriate enzyme.

3. The method according to claim 2, wherein the appropriate enzyme is collagenase.

4. The method according to claim 1, further comprising the step c) of encapsulating the pancreatic islets of step b) in a device.

5. The method according to claim 4, wherein the device is a protective device comprising:
(i) a semi-permeable membrane of high polymer;
(ii) a mesh reinforcement; and
(iii) a microcapsule, a microparticle or a mixture thereof;
or comprising:
(i) a semi-permeable membrane of high polymer; and
(ii) a microcapsule, a microparticle or a mixture thereof.

6. The method according to claim 1, wherein the pancreatic endocrine cells of step a) comprise beta cells, or wherein the pancreatic endocrine cells of step a) comprise beta cells and alpha cells.

7. The method according to claim 1, wherein the immature canine pancreas is an immature dog pancreas.

8. The method according to claim 1, wherein the immature canine pancreas is a foetal canine pancreas, or a neonatal canine pancreas, or is obtained from a non-adult canine.

9. The method according to claim 8, wherein the fetal canine pancreas is in the last third of gestation.

10. The method according to claim 8, wherein the fetal canine pancreas is at days 40 to 60 post conception.

11. Canine pancreatic islets obtainable by the method according to claim 1.

12. The canine pancreatic islets according to claim 11, wherein said islets possess at least one feature selected from of:
presence of canine alpha cells;
presence of canine beta cells;
expression of canine-specific insulin;
expression of canine-specific glucagon; and
any combination thereof.

13. (canceled)

14. The canine pancreatic islets according to claim 11, wherein said pancreatic islets are positive to reaction with canine-specific anti-insulin, canine-specific anti-glucagon, anti-GAD and/or anti-IA2 antibodies.

15. The canine pancreatic islets according to claim 11, wherein said pancreatic islets are capable of secreting canine specific insulin and/or canine specific glucagon in response to glucose stimulation.

16. (canceled)

17. A culture comprising canine pancreatic islets according to claim 11 in an appropriate culture medium.

18. A veterinary composition comprising a pharmaceutically acceptable carrier and an effective amount of the canine pancreatic islets according to claim 11.

19. Method for reducing the risk of developing or treating a canine pancreatic disorder in an animal, comprising the administration of the canine pancreatic islets according to claim 11, or a veterinary composition comprising a pharmaceutically acceptable carrier and an effective amount of the canine pancreatic islets according to claim 11.

20. The method according to claim 19, wherein said canine pancreatic disorder is canine diabetes.

21. The method according to claim 19, wherein said canine pancreatic islets or said veterinary composition are (is) transplanted in the pancreas, the liver, a muscle, a subcutaneous tissue, the renal subcapsule, or the peritoneal cavity of said animal.

22. The method according to claim 19, wherein said canine pancreatic islets or said veterinary composition are (is) administered by injection in said animal.

23-32. (canceled)

33. A method for preparing transduced canine pancreatic islets, transduced canine pancreatic beta cells or canine beta cell tumours comprising the step of:
a) transducing or co-transducing the canine pancreatic islets of claim 11 with i) a lentiviral vector expressing SV40 Large T antigen under the control of the insulin promoter, or ii) with a lentiviral vector expressing SV40 Large T antigen under the control of the insulin promoter and a lentiviral vector expressing hTert under the control of the insulin promoter, or iii) a lentiviral vector expressing both SV40 Large T antigen and hTert under the control of the insulin promoter.

34. The method according to claim 33, further comprising the step of:
b) collecting the canine pancreatic islets obtained at step a) to form a homogenous transduced canine islet population.

35. The method according to claim 33, further comprising the step of:
b) dissociating the transduced pancreatic beta cells from the transduced canine pancreatic islets of step a); and
c) harvesting the pancreatic beta cells contained in the dissociated islets of step b), to form a homogenous transduced canine pancreatic beta cell population.

36. The method according to claim 33, further comprising the steps of:
b) introducing the transduced pancreatic islets obtained in a) into the kidney capsule of a first severe combined immunodeficiency (scid) non-human animal;
c) allowing the transduced pancreatic islets to develop insulinoma-like structures, wherein the canine pancreas cells in insulinoma-like structures have differentiated to insulin-producing pancreatic islets and/or beta cells;
d) micro-dissecting the insulinoma-like structures obtained in step c), and dissociating the islets and/or cells thereof;
e) sub-transplanting the islets and/or cells obtained in step d) into the kidney capsule of a second scid non-human animal;
f) allowing the sub-transplanted islets and/or cells in step e) to develop and regenerate newly developed insulinoma-like structures, wherein said newly developed insulinoma-like structures are enriched in insulin-producing pancreatic islets and/or beta cells; and
g) micro-dissecting the insulinoma-like structures obtained in step f), and dissociating and collecting the islets and/or cells thereof.

37-49. (canceled)

50. Canine pancreatic islets, canine beta cell tumours or canine pancreatic beta cells obtainable by the method according to claim 33.

51. The canine pancreatic islets, canine beta cell tumours or canine pancreatic beta cells according to claim 50, wherein said tumours or cells have at least one feature selected from:
Carboxypeptidase-A negative
transcriptional factor Pdx1 positive
transcription factor MafA positive
proconvertase Pcsk1 positive
expression of Glucose transporter Glut2
expression of Kenj11 and Abcc8 coding for subunits of the potassium channel
expression of zinc transporter Znt8 (Slc30a8)
expression of canine-specific insulin
positive to reaction with canine-specific anti-insulin, anti-GAD and/or anti-IA2 antibodies and
any combination thereof.

52-73. (canceled)
